(19) United States
(12) Patent Application Publication
(10) Pub. No.: US 2024/0148665 A1
(43) Pub. Date: May 9, 2024

(54) SUGAR-CONJUGATED LIPID NANOPARTICLES AND METHODS OF USE THEREFOR

(71) Applicant: University of Virginia Patent Foundation, Charlottesville, VA (US)

(72) Inventors: Anuradha Illendula, Crozet, VA (US); Samir Zuberi, Springfield, VA (US); Alex Powell, Williamsburg, VA (US)

(21) Appl. No.: 18/495,741

(22) Filed: Oct. 26, 2023

Related U.S. Application Data
(63) Continuation-in-part of application No. PCT/US2023/066270, filed on Apr. 26, 2023.
(60) Provisional application No. 63/335,142, filed on Apr. 26, 2022.

(51) Int. Cl. A61K 9/51; A61K 31/7105; A61K 47/10; A61K 47/24; A61K 47/26; A61K 47/28

(52) U.S. Cl. CPC A61K 9/5123; A61K 31/7105; A61K 47/10; A61K 47/24; A61K 47/26; A61K 47/28

(57) ABSTRACT

Provided are compositions that include compositions of galactosyl-conjugated lipid nanoparticles (LNPs) encapsulating one or more active agents. In some embodiments, the galactosyl-conjugated LNPs have a lipid component having D-Lin-MC3-DMA, ALC-0315 and SM-102, cholesterol. DSPC and DOPE, and DMG-2000-PEG. In some embodiments, the GalNAc-conjugated LNP has one or more galactosyl moieties bioconjugated to cholesterol present with a lipid component of the GalNAc-conjugated LNP. Also provided are methods for treating and/or preventing diseases, disorders, and/or conditions associated with undesirable PCSK9 gene expression, optionally a cardiovascular disease, disorder, or condition, including but not limited to atherosclerosis and/or thrombosis, and sepsis, septic shock, cytokine storm, or sequelae thereof.

### CROSS REFERENCE TO RELATED APPLICATION

The present application is a continuation-in-part of PCT International Patent Application Serial No. PCT/US2023/066270, filed Apr. 26, 2023 (pending), which itself claims the benefit of U.S. Provisional Patent Application Ser. No. 63/335,142, filed Apr. 26, 2022 (expired), the disclosure of each of which is incorporated herein by reference in its entirety.

### REFERENCE TO SEQUENCE LISTING XML

The Sequence Listing XML associated with the instant disclosure has been electronically submitted to the United States Patent and Trademark Office via the Patent Center as a 9,935 byte UTF-8-encoded XML file created on Oct. 26, 2023 and entitled “3062_186_2_CIP.xml”, The Sequence Listing submitted via Patent Center is hereby incorporated by reference in its entirety.

### TECHNICAL FIELD

The presently disclosed subject matter relates generally to methods for employing compositions comprising sugar-conjugated lipid nanoparticles to deliver siRNA to target cells, tissues, and organs in order to treat and/or prevent diseases, disorders, and conditions associated with undesirable levels of PCSK9 gene expression.

### BACKGROUND

Recently, there has been an increase in the number of oligonucleotide-based therapies that seek to alter specific gene and protein expression for various cancers, cardiovascular diseases, neurological diseases, and oilier conditions. Direct bioconjugated and chemically modified nucleic acid therapeutics induce chronic effects but are limited by high treatment costs ($300,000 to $2.1 million/year). While these therapies are gaining popularity, there are still many hurdles to producing them as they rapidly degrade in vivo, particularly for “unmodified” nucleic acid-based drugs. Adenoviral and lentiviral formulations have been explored extensively in the gene therapy space but are known to stimulate unwanted immune responses on top of high manufacturing costs and ethical concerns (Wang et al., 2019; Hu et al., 2020; Zhang et al., 2022: Zhu et al, 2022). These viral modalities are therefore not sufficiently safe and efficient for use in the treatment of conditions requiring high-dose systemic administration and/or multiple dosing regimens.

Additionally, a key challenge in the development of nucleic acid-based gene therapies (e.g., with siRNA, mRNA, and/or miRNA) is the development of an efficient method of targeted, intracellular delivery. To address these needs, disclosed herein are engineered and validated nanoparticle approaches that achieve selective liver targeting while “shielding” the recipient from potentially immunogenic, toxic, and metabolizable nucleic acid (e.g., siRNA) packages. Specifically, a hexose monosaccharide-conjugated entrapped lipid nanoparticle (LNP) for hepatocellular delivery that delivers nucleic acid including but not limited to siRNA is disclosed. The exemplary bioconjugated galactosyl-cholesterol intercalated LNP disclosed herein utilizes the targeted moiety as a novel component of the LNP. As proof of concept, the bioactive PCSK9 siRNA component of Inclisiran (no GalNAc, L96) was encapsulated within the LNP with the targeting motif, galactosyl, on the outside of the LNP.

PCSK9 is a protein that is present in the bloodstream and acts as a suicide inhibitor to low density lipoprotein (LDL) molecules by irreversibly binding to LDL receptors (LDLRs) on the cell membrane. This causes the complex to be endocytosed and transported to the lysosome, where the receptor is degraded. This process severely inhibits a cell's ability to bring in LDL particles from the bloodstream and digest them as without bound PCSK9, LDLRs are recycled allowing them to further metabolize cholesterol. The main therapies for improving LDL digestion include statins, cholesterol uptake inhibitors, and now PCSK9 inhibitors. Statins inhibit cholesterol synthesis within the cell through binding to and inhibiting HMG-CoA reductase. This promotes uptake of cholesterol from outside the cell and thus helps to improve the productivity of the cell's functioning LDLRs and absorption of more LDL particles from the bloodstream. Statins have significant side effects as they prevent the creation of many enzymes involved in the cholesterol synthesis pathway. This therapy does not work for those with any of various known mutations that prevent LDLRs from functioning. These patients must use cholesterol uptake inhibitors, which prevent absorption of any cholesterol from the small intestine/digestive system. This prevents LDL buildup within the bloodstream as most of the cholesterol brought in through the diet is excreted as waste. Finally, PCSK9 inhibition has recently become a new method of promoting LDL uptake. However, the main methods that have been developed thus far include using modified siRNA therapies like Inclisiran and the use of antibodies, alirocumab and evolocumab, which target PCSK9 mRNA and proteins respectively, binding to and degrading them. Both of these therapies are very expensive and antibody treatment eventually loses efficacy as the body develops an immune response toward them, so better treatment options still must be developed (Lagace, 2014; Lodish et al., 2021; Arnold & Koenig, 2022). This platform can be adopted to target many receptors including but not limited to Lectins, C-Lectin, L/E/P-selectin, and/or Transferrin, in sonic embodiments by using different sugars including but not limited to galactose, N-acetyl galactosamine, glucose. N-acetyl glucosamine, mannose, trehalose, fucoidan, pyranose, and furanose,

### SUMMARY

This Summary lists several embodiments of the presently disclosed subject matter, and in many cases lists variations and permutations of these embodiments of the presently disclosed subject matter. This Summary is merely exemplary of the numerous and varied embodiments. Mention of one or more representative features of a given embodiment is likewise exemplary. Such an embodiment can typically exist with or without the features) mentioned; likewise, those features can be applied to other embodiments of the presently disclosed subject matter, whether listed in this Summary or not. To avoid excessive repetition, this Summary does not list or suggest all possible combinations of such features.

The presently disclosed subject matter relates in some embodiments to compositions comprising, consisting essentially of, or consisting of sugar-conjugated lipid nanoparticles (LNPs) with one or more active agents encapsulated therein, optionally wherein the sugar is a hexose monosaccharide. In some embodiments, the sugar is selected from the group consisting of mono-, di-, and triannary galactosyl, N-acetyl galactosamine (GalNac), glucose, N-acetyl glucosamine (GluNac), mannose, trehalose, and fucoidan, pyranose, and furanose. In some embodiments, the galactosyl conjugated LNP further comprises a lipid component comprising about 5-10% cholesterol. In some embodiments, the galactosyl conjugated LNP comprises a lipid component comprising D-Lin-MC3-DMA, cholesterol, DSPC, DOPE and DMG-2000-PEG, In some embodiments, the galactosyl conjugated LNP comprises a lipid component comprising of ALC-0315, cholesterol, DSPC, DOPE, and DMG-2000-PEG. In some embodiments, the galactosyl conjugated LNP comprises a lipid component comprising of SM-102, cholesterol, DSPC, DOPE, and DMG-2000-PEG. In some embodiments, the galactosyl conjugated LNP comprises a combination of lipid components comprising D-Lin-MC3-DMA and ALC-0315, cholesterol, DSPC, DOPE, and DMG-2000-PEG. In some embodiments, the galactosyl conjugated LNP comprises a combination of lipid components comprising D-Lin-MC3-DMA and SM-102, cholesterol, DSPC, DOPE, and DMG-2000-PEG. In some embodiments, the galactosyl conjugated LNP comprises one or more galactosyl moieties bioconjugated cholesterol present with a lipid component of the galactosyl conjugated LNP. In some embodiments, the active agent is an inhibitory nucleic acid, optionally an siRNA and/or a plasmid. In some embodiments, the inhibitory nucleic acid inhibits a biological activity of a PCSK9 gene product, optionally a human PCSK9 gene product, and further optionally wherein the inhibitory nucleic acid is an inclisiran derivative lacking its GalNAc moiety. In some embodiments, the galactosyl conjugated LNP targets the active agent to a cell, tissue, or organ of interest, optionally wherein the cell is a hepatocyte and/or the organ is liver. In some embodiments, the LNP comprises a ratiometric combination of ionizable lipids. In some embodiments, the LNP comprises a ratiometric combination of ionizable lipids with a specific dissociation constant value of between about 6.05 and about 6.44. In some embodiments, the LNP comprises one or more mono-, di-, and/or ternary hydrophobic fatty acids and/or one or more hydrophilic polyethylene glycols conjugated to the sugar moiety onto cholesterol at its head or tail and/or onto a PEG moiety. In some embodiments, at least one of the one or more fatty acids and/or the polyethylene glycol comprises a chain length of 1 to 15 carbons.

In some embodiments, the presently disclosed subject matter also relates to methods for treating and/or preventing diseases, disorders, and/or conditions associated with undesirable gene expression. In some embodiments, the methods comprise, consist essentially of, or consist of administering to a subject in need thereof an effective amount of a composition as disclosed herein, wherein the composition comprises an active agent that inhibits the activity of the gene with which the disease, disorder, or condition is associated. In some embodiments, the disease, disorder, and/or condition is selected from the group consisting of hypercholesterolemia, hepatocytes with hexose sugar conjugation to asialoglycoprotein receptor 1 (ASGPR); hepatocellular carcinoma (HCC) including but not limited to MC stages I-III, nonalcoholic fatty liver disease (NAFLD), nonalcoholic steatohepatitis (NASH), hepatic cirrhosis, chronic active hepatitis, hepatitis resulting from infection with one or more of hepatitis viruses A-E, and hepatocellular hepatitis resulting from infection with Epstein Barr virus.

In some embodiments, the presently disclosed subject matter also relates to methods for targeting active agents to hepatocytes. In some embodiments, the methods comprise, consist essentially of, or consist of contacting the hepatocyte with a composition comprising an active agent encapsulated by a galactosyl (including but not limited to N-acetylgalactosamine (GalNAc)) conjugated lipid nanoparticle (LNP), wherein the galactosyl LNP comprises a lipid component comprising D-Lin-MC3-DMA, cholesterol, DSPC, and DMG-2000-PEG, optionally wherein the cholesterol comprises one or more galactosyl moieties conjugated thereto, whereby the active agent is targeted to the hepatocyte. In some embodiments, the active agent is an inhibitory nucleic acid, optionally an siRNA, In some embodiments, the inhibitory nucleic acid inhibits a biological activity of a gene that is expressed in the hepatocyte. In some embodiments, the gene that is expressed in the hepatocyte is an PCSK9 gene, optionally a human PCSK9 gene, and further optionally wherein the inhibitory nucleic acid is an inclisiran derivative lacking its GalNAc moiety. In some embodiments, the active agent inhibits expression of the gene in the hepatocyte to thereby treat and/or prevent a disease, disorder, or condition associated with undesirable expression of the gene in the hepatocyte. In some embodiments, the gene is an PCSK9 gene, optionally a human PCSK9 gene, and further wherein the composition treats and/or prevents hypercholesterolemia in the subject.

In some embodiments, the presently disclosed subject matter also relates to methods for treating and/or preventing a disease, disorder, or condition associated with undesirable preprotein convertase subtilisin/kexin type 9 (PCSK9) gene expression, the method comprising, consisting essentially of, or consisting of administering to a subject in need thereof an effective amount of a comprising a sugar-conjugated lipid nanoparticle (LNP) and an active agent encapsulated therein, optionally wherein the sugar is a hexose monosaccharide, wherein the active agent modulates the PCSK9 gene expression to thereby treat and/or prevent the disease, disorder, or condition. In some embodiments, the sugar is selected from the group consisting of mono-, di-, and triannary galactosyl, N-acetyl galactosamine (GalNac), glucose, N-acetyl glucosamine (GluNac), mannose, trehalose, fucoidan, pyranose, and furanose. In some embodiments, the LNP is a galactosyl-conjugated LNP, and the galactosyl-conjugated LNP further comprises a lipid component comprising about 5-10% cholesterol. In some embodiments, the LNP, optionally the galactosyl-conjugated LNP, comprises a lipid component comprising one or more of hydrolyzable ionizable lipids, optionally selected from the group consisting of D-Lin-MC3-DMA, ALC-0315, and SM-102; cholesterol and/or an analog thereof, optionally P-sitosterol; and optionally one or more helper lipids, optionally selected from the group consisting of DSPC, DOPE, and DMG-2000-PEG, further optionally two, three, or all four of D-Lin-MC3-DMA, cholesterol, DSPC, and DMG-2000-PEG.

In some embodiments of the presently disclosed methods, the LNP is a galactosyl-conjugated LNP that comprises one or more galactosyl moieties bioconjugated to cholesterol present with a lipid component of the galactosyl-conjugated LNP.

In some embodiments of the presently disclosed methods, the active agent is a nucleic acid, optionally an inhibitory nucleic acid, further optionally an siRNA. In some embodiments, the inhibitory nucleic acid inhibits a biological activity of a PCSK9 gene product, optionally a human PCSK9 gene product, and further optionally wherein the inhibitory nucleic acid is an inclisiran derivative lacking a GalNAc moiety.

In some embodiments of the presently disclosed methods, the LNP, optionally the galactosyl-conjugated LNP, targets the active agent to a cell, tissue, or organ of interest, optionally wherein the cell tissue, or organ of interest is selected from the group consisting of vasculature or a vasculature cell, optionally a vascular endothelial cell, a muscle cell, a fibroblast, and/or a non-vascular endothelial cell.

In some embodiments, the LNP comprises a ratiometric combination of ionizable lipids. In some embodiments, the LNP comprises a ratiometric combination of ionizable lipids with a specific dissociation constant value of between about 6.05 and about 6.44.

In some embodiments, the LNP comprises one or more mono-, di-, and/or ternary hydrophobic fatty acids and/or one or more hydrophilic polyethylene glycols conjugated to the sugar moiety onto cholesterol at its head or tail and/or onto a polyethylene glycol (PEG) moiety. In some embodiments, at least one of the one or more fatty acids and/or the PEG moiety comprises a chain length of 1 to 15 carbons.

In some embodiments of the presently disclosed subject matter, the disease, disorder, or condition is selected from the group consisting of an inflammatory disease, disorder, or condition; a cardiovascular disease, disorder, or condition, optionally atherosclerosis and/or thrombosis; and sepsis, septic shock, cytokine storm, or sequelae thereof.

In some embodiments, the presently disclosed methods further comprise administering to the subject in need thereof an effective amount of a second active agent that is appropriate for treating the disease, disorder, or condition. In some embodiments, the disease, disorder, or condition is an inflammatory disease, disorder, or condition, and the second active agent is an anti-inflammatory agent, optionally a nonsteroidal anti-inflammatory drug (NSAID), a steroid, acetaminophen or other COX-3 inhibitor, a 5-lipoxygenase inhibitor, a leukotriene receptor antagonist, a leukotriene A4 hydrolase inhibitor, an antihistatninic, a histamine 2 receptor antagonist, a phosphodiesterase-4 antagonist, a cytokine antagonist, a CD44 antagonist, an antineoplastic agent, a 3-hydroxy-3-methyldutaryl coenzyme A inhibitor (e.g., a statin), an estrogen, an androgen, an antiplatelet agent, an antidepressant, a Helicobacter pylori inhibitor, a proton pump inhibitor, a thiazolidinedione, or a dual-action compound. In some embodiments, the disease, disorder, or condition is a cardiovascular disease, disorder, or condition, optionally atherosclerosis, and the second active agent is an acyl-coenzyme A: cholesterol acyltransferase (ACAT) inhibitor, including but not limited to a selective inhibitor of ACAT-1, ACAT-2 as well as dual inhibitors of ACAT-1 and ACAT-2; an alpha-adrenergic blocking drug (alpha-blocker), an alpha/beta blocker, an angiotensin-converting enzyme (ACE) inhibitor, an aldosterone antagonist, an angiotensin II receptor antagonist, an anti-arrhythmic, an anticoagulant, an antiplatelet agent, an apolipoprotein A-1 (apoA-1) mimetic, a beta-blocker, a bile acid sequestrant, a calcium-channel blocker, an ApoB cholesteryl ester transfer protein (CETP) inhibitor, a cholesterol absorption inhibitor, a diuretic, a dyslipidcmia agent, an endothelin receptor antagonist, a fibrate, a 3-hydroxy-3-methyl-glutaryl-coenzyme A (HMG-CoA) reductase inhibitor, an LCAT activator, an LDL receptor inducer, a lipase inhibitor, a lipoprotein-associated phospholipase A2 (Lp-PLA2) inhibitor, a microsomal triglyceride transfer protein (MTP) inhibitor, a platelet aggregation inhibitor, a PPAR agonist and/or activator, optionally a PPARγ agonist, PPARα agonist, and/or a PPAR dual α/γ agonists, a second PCSK9 antisense nucleic acid or RNAi, a squalene epoxidase inhibitor, a squalene synthetase inhibitor, a thrombolytic, a thyroid receptor beta activator, or any combination thereof. In some embodiments, the disease, disorder, or condition is a cardiovascular disease, disorder, or condition, and the second active agent targets a vasculature cell, a muscle cell, a fibroblast, and/or an endothelial cell, optionally a vascular endothelial cell, wherein the second active agent binds to a target selected from the group consisting of LOX-1, a selectin, optionally a P-selectin or an E-selectin, an integrins, and a neuropilin. In some embodiments, the disease, disorder, or condition is sepsis, septic shock, cytokine storm or a sequalae thereof and the second active agent is an antibiotic, a complement inhibitor, optionally a complement inhibitor selected from the group consisting of a C5a inhibitor, a C5aR inhibitor, a C3 inhibitor, optionally wherein the C3 inhibitor is Compstatin, a Compstatin analog, a Compstatin peptidomitnetic, or a Compstatin derivative, a factor D inhibitor, a factor B inhibitor, a C4 inhibitor, a C1q inhibitor, or any combination thereof.

In some embodiments, the presently disclosed subject matter also relates to methods for targeting active agents to hepatocytes. In some embodiments, the methods comprise, consist essentially of, or consist of contacting the hepatocyte with a composition comprising an active agent encapsulated by a sugar-conjugated lipid nanoparticle (LNP), optionally a hexose monosaccharide-conjugated LNP, further optionally an N-acetylgalactosamine (GalNAc) conjugated lipid nanoparticle (LNP), wherein the sugar-conjugated LNP comprises a lipid component comprising ionizable aminolipid ALC-0315 (6-((2-hexyldecanoyl)oxy)-N-(6-((2-hexyldecanoyl)oxy)hexyl)-N-(4-hydroxybutyl)hexan-1-aminium) and one or more of D-Lin-MC3-DMA, cholesterol, DSPC, DOPE, and DMG-2000-PEG, optionally two, three, four, or all five of D-Lin-MC3-DMA, cholesterol, DSPC, and DMG-2000-PEG, further optionally wherein the lipid component comprises cholesterol with one or more GalNAc moieties conjugated thereto, optionally with 25% of the cholesterol being galactosyl-cholesterol, whereby the active agent is targeted to the hepatocyte. In some embodiments, the LNP comprises 1, 2, 3, 4, 5, 6, 7, 8, 9, or 10 parts ALC-0315 to 1 part DLin-MC3-DMA, cholesterol, 25% of which is galactosyl-cholesterol, DOPE, and DSPC. In some embodiments, the active agent comprises an inhibitory nucleic acid that inhibits a biological activity of a PCSK9 gene product that is expressed in the hepatocyte. In some embodiments, the wherein the anti-PCSK9 inhibitory nucleic acid is an anti-PCSK9 siRNA. In some embodiments, the anti-PCSK9 inhibitory nucleic comprises a sense strand comprising SEQ NO: 7, an antisense strand comprising SEQ NO: 8, or both. In some embodiments, the PCSK9 gene product is a human PCSK9 gene product. In some embodiments, the active agent inhibits expression of the PCSK9 gene in the hepatocyte to thereby treat and/or prevent a disease, disorder, or condition associated with undesirable expression of the PCSK9 gene.

Accordingly, it is an object of the presently disclosed subject matter to provide compositions comprising, consisting essentially of, or consisting of galactosyl conjugated lipid nanoparticles (LNPs) with one or more active agents encapsulated therein. This and other objects are achieved in whole or in part by the presently disclosed subject matter.

Further, an object of the presently disclosed subject matter having been stated above, other objects and advantages of the presently disclosed subject matter will become apparent to those skilled in the art after a study of the following description, Figures, and EXAMPLE.

### BRIEF DESCRIPTION OF THE FIGURES

FIG. 1A: Dynamic light scattering (DLS) of 0%, 25%, and 50% Gal-Chol LNPs encapsulating a PCSK9 siRNA.

FIG. 1B: Zeta-potential of 0%, 25%, and 50% Gal-Chol LNPs.

FIG. 2A: DLS of 0%, 25%, and 50% Gal-Chol ghost (no oligonucleotide) LNPs.

FIG. 2B: Zeta-potential of 0%, 25%, and 50% Gal-Chol ghost LNPs.

FIG. 3A: DLS of ½ PEG, SM-102, and ALC-0315 LNPs encapsulating GFP plasmid.

FIG. 3B: Zeta-potential of ½ PEG, SM-102, and ALC-0315 LNPs.

FIGS. 3C and 3D: DLS (FIG. 3C) and Nanoparticle Tracking Analysis (NTA; FIG. 3D) data of ALC 1:1 Dlin 25% galactosyl-cholesterol LNP verifying the NP diameter through a second characterization technique. The discrepancy is due to differences is the methods used to determine NP size.

FIGS. 3E-3H: DLS of ALC N/P 6 LNP with no galactosyl (FIG. 3E), ALC N/P 8 LNP with no galactosyl (FIG. 3F), LNP 0% Gal DSPC:DOPE 8:2 (FIG. 3G), and LNP 25% Gal DSPC:DOPE 8:2 (FIG. 3H).

FIG. 31: Cryo-electron microscopy of LNPs to visualize NP morphology and size distributions. These images show a relatively monodisperse LNP both in ghost and siRNA loaded NPs.

FIG. 4: Bar graphs showing comparison between the siRNA concentrations between N/P 2 (top) and N/P 4 (bottom) to show its effect of siRNA concentrations. siRNA concentrations were calculated using the RIBOGREEN® assay. Ratios refer to the lipid stated and Dlin-MC3-DMA. encapsulation efficiency of LNPs.

FIG. 5: Flow Cytometry analysis of Cy3-GAPDH siRNA encapsulated LNPs demonstrating equitable siRNA distribution per LNP and high particle concentration in 50 μL sample. (Left panel) Flow cytometry gate displaying LNP singlets. (Right Panel) Flow cytometry gate displaying Cy3 signal from Cy3-GAPDH siRNA encapsulated LNPs.

FIG. 6: Exemplary dosing scheme for LNPs on adherent cells.

FIG. 7A: Western blot of AML12 cells, harvested 48 hours post dose.

FIG. 7B: Bar graph of quantification of the Western blot data in FIG. 7A, data normalized to total protein.

FIG. 8A: Western blot of HepG2 cells treated with 300 ng siRNA/mL media. Cells harvested 48 hours post dose.

FIG. 8B: Bar graph of quantification of the Western blot data in FIG. 8A, data normalized to total protein. No Gal, non-significant Gal-Chol 25%, p<0.0034; 50% gal-chol, p<0.0049.

FIG. 8C: Western blot of HepG2 incubated for 48 hours with PCSK9 siRNA LNPs with N/P 4, varying lipid ratios in order to modulate the pKa values. Western blot was normalized to total protein stain.

FIG. 8D: Bar graph of quantification of the Western blot data in FIG. 8C, data normalized to total protein.

FIG. 9: A bar graph showing RT-qPCR analysis of RepG2s incubated for 48 hours with FIDAC2 (as a proof of concept) siRNA LNPs (300 ng siRNA/mL media) using B2M as a control reference gene. All samples are normalized with respect to the negative control (no treatment). ALC, SM titles refer to LNPs with 1:1 Dlin:ALC/SM. These data correlated with the known optimal pKa range for transfection, showing improved transfection with the additions of SM-102 and ALC-0315.

FIG. 10: A series of graphs of pKa and siRNA concentrations of formulations while modulating the ratio of Dlin-MC3-DMA and ALC-0315 or SM-102. The discrepancy in trends between the siRNA concentrations and pKa were due to the change of lipids used in order to modulate the pKa. 1:1 SM/ALC was used to show transfection because its pKa is within the optimal rage for hepatocellular transfection.

FIG. 11: Schematic representation of synthesis of sugar, substituted sugar conjugated cholesterol linked by fatty acid chain, glycol linker and/or amide linker. R1=R2=R3 are equal or different substitution like H and sugar; n=number of repeated units.

FIG. 12: Schematic representation of one mechanism by which sepsis can lead to cytokine storm and consequences thereof, and how LNPs of the presently disclosed subject matter can be used to prevent or treat cytokine storm and its consequences.

FIG. 13: Schematic representation of how cardiovascular diseases (CVDs) develop with respect to PCSK9. Briefly, PCSK9 can induce inflammation through the NLRP3 inflammasome and platelet activation. Inflammation can cause macrophages to differentiate into foam cells, causing atherosclerosis. Similarly, high PCSK9 concentrations can cause platelet activation and, thus, blood clots (i.e., thrombosis). These effects can be due to PCSK9-mediated inflammation. Because the LNPs of the presently disclosed subject matter inhibit PCSK9 production at the source, and in some embodiments a second complementary drug can inhibit one or more converging pathways in the target cells, tissues, and organs of interest, the combinations can alleviate these diseased outcomes.

FIG. 14: Schematic representation that is complementary to the FIG. 13. In FIG. 14, PCSK9 is shown to decrease LDLR, rendering a higher concentration of PCSK9 in circulation (in some embodiments, by degradation of a binding partner) and hindering the clearance of LDL via the LDL receptor (LDLR). PCSK9 can also bind to CD36, which can activate the LOX-1 pathway, LDL circulation, therefore, has a greater chance of becoming oxidized. Oxidized LDL can bind to and activate the LOX-1 pathway, which can causes reactive oxygen species (ROS) production and inflammation. This inflammation can give rise to CVD because it can cause the differentiation of macrophages (atherosclerosis/CVD), the cytokine storm (sepsis), and increase the production of pro-coagulant factors (CVD/thrombosis). The LNPs of the presently disclosed subject matter can decrease PCSK9 production, and in some embodiments, a nano-NLRP3 inhibitor can inhibit the inflammasome in the vasculature to prevent and/or treat related disease.

### DETAILED DESCRIPTION

### I. Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the presently disclosed and claimed subject matter. Following long-standing patent law convention, the terms “a”, “an”, and “the” refer to “one or more” when used in this application, including in the claims. For example, the phrase “an antibody” refers to one or more antibodies, including a plurality of the same antibody. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and greater than 100.

Unless otherwise indicated, all numbers expressing quantities of ingredients, reaction conditions, and so forth used in the specification and claims are to be understood as being modified in all instances by the term “about”. The term “about”, as used herein when referring to a measurable value such as an amount of mass, weight, time, volume, concentration, or percentage, is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods and/or employ the disclosed compositions. Accordingly, unless indicated to the contrary, the numerical parameters set forth in this specification and attached claims are approximations that can vary depending upon the desired properties sought to be obtained by the presently disclosed subject matter.

A disease or disorder is “alleviated” if the severity of a symptom of the disease, condition, or disorder, or the frequency at which such a symptom is experienced by a subject, or both, are reduced.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D.

The terms “additional therapeutically active compound” and “additional therapeutic agent”, as used in the context of the presently disclosed subject matter, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease, or disorder being treated.

As used herein, the term “adjuvant” refers to a substance that elicits an enhanced immune response when used in combination with a specific antigen.

As use herein, the terms “administration of” and/or “administering” a compound should be understood to refer to providing a compound of the presently disclosed subject matter to a subject in need of treatment.

The term “comprising”, which is synonymous with “including” “containing”, or “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting essentially of” limits the scope of the related disclosure or claim to the specified materials and/or steps, plus those that do not materially affect the basic and novel characteristic(s) of the disclosed and/or claimed subject matter. For example, a pharmaceutical composition can “consist essentially of” a pharmaceutically active agent or a plurality of pharmaceutically active agents, which means that the recited pharmaceutically active agent(s) is/are the only pharmaceutically active agent(s) present in the pharmaceutical composition. It is noted, however, that carriers, excipients, and/or other inactive agents can and likely would be present in such a pharmaceutical composition, and are encompassed within the nature of the phrase “consisting essentially of”.

As used herein, the phrase “consisting of” excludes any element, step, or ingredient not specifically recited. It is noted that, when the phrase “consists of” appears in a clause of the body of a claim, rather than immediately following the preamble, it limits only the element set forth in that clause; other elements are not excluded from the claim as a whole.

With respect to the terms “comprising”, “consisting of” and “consisting essentially of”, where one of these three terms is used herein, the presently disclosed and claimed subject matter can include the use of either of the other two terms. For example, a composition that in some embodiments comprises a given active agent also in some embodiments can consist essentially of that same active agent, and indeed can in some embodiments consist of that same active agent.

As use herein, the terms “administration of” and or “administering” a compound should be understood to mean providing a compound of the presently disclosed subject matter or a prodrug of a compound of the presently disclosed subject matter to a subject in need of treatment.

As used herein, an “agent” is meant to include something being contacted with a cell population to elicit an effect, such as a drug, a protein, a peptide. An “additional therapeutic agent” refers to a drug or other compound used to treat an illness and can include, for example, an antibiotic or a chemotherapeutic agent.

As used herein, an “agonist” is a composition of matter which, when administered to a mammal such as a human, enhances or extends a biological activity attributable to the level or presence of a target compound or molecule of interest in the mammal.

An “antagonist” is a composition of matter which when administered to a mammal such as a human. inhibits a biological activity attributable to the level or presence of a compound or molecule of interest in the mammal.

As used herein, “alleviating a disease or disorder symptom”, means reducing the severity of the symptom or the frequency with which such a symptom is experienced by a patient, or both.

As used herein, an “analog” of a chemical compound is a compound that, by way of example, resembles another in structure but is not necessarily an isomer (e.g., 5-fluorouracil is an analog of thymine).

As used herein, amino acids are represented by the full name thereof, by the three-letter code corresponding thereto, and/or by the one-letter code corresponding thereto, as summarized in the following Table:

### Amino Acid Codes and Functionally Equivalent Codons

| | 3- | 1- | |
|---|---|---|---|
| | Letter | Letter | |
| Full Name | Code | Code | Functionally Equivalent Codons |
| Aspartic Acid | Asp | D | GAC; GAU |
| Glutamic Acid | Glu | E | GAA; GAG |
| Lysine | Lys | K | AAA; AAG |
| Arginine | Arg | R | AGA; AGG; CGA; CGC; CGG; CGU |
| Histidine | His | H | CAC; CAU |
| Tyrosine | Tyr | Y | UAC; UAU |
| Cysteine | Cys | C | UGC; UGU |
| Asparagine | Asn | N | AAC; AAU |
| Glutamine | Gln | Q | CAA: CAG |
| Serine | Ser | S | ACG; AGU; UCA; UCC; UCG; UCU |
| Threonine | Thr | T | ACA; ACC; ACG; ACU |
| Glycine | Gly | G | GGA; GGC; GGG; GGU |
| Alanine | Ala | A | GCA; GCC; GCG; GCU |
| Valine | Val | V | GUA; GUC; GUG; GUU |
| Leucine | Leu | L | UUA; UUG; CUA; CUC; CUG; CUU |
| Isoleucine | Ile | 1 | AUA; AUC; AUU |
| Methionine | Met | M | AUG |
| Proline | Pro | P | CCA; CCC; CCG; CCU |
| Phenylalanine | Phe | F | UUC; UUU |
| Tryptophan | Trp | W | UGG |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source, As used herein, “synthetic amino acid” also encompasses chemically modified amino acids, including but not limited to salts, amino acid derivatives (such as amides), and substitutions. Amino acids contained within the compositions of the presently disclosed subject matter, and particularly at the carboxy- or amino-terminus, can be modified by methylation, amidation, acetylation or substitution with other chemical groups which can change the peptide's circulating half-life without adversely affecting their activity, Additionally, a disulfide linkage may be present or absent in the compositions of the presently disclosed subject matter.

The term “amino acid” is used interchangeably with “amino acid residue” and may refer to a free amino acid and to an amino acid residue of a peptide. It will be apparent from the context in which the term is used whether it refers to a free amino acid or a residue of a peptide.

Amino acids have the following general structure:

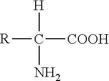

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the presently disclosed subject matter follows the conventional practice wherein the amino group is presented to the left and the carboxy group to the right of each amino acid residue. In the formulae representing selected specific embodiments of the presently disclosed subject matter, the amino-and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

The term “antibody” as used herein refers to an immunoglobulin molecule which is able to specifically or selectively bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the presently disclosed subject matter exist in a variety of forms. The term “antibody” refers to polyclonal and monoclonal antibodies and derivatives thereof (including chimeric, synthesized, humanized and human antibodies), including an entire immunoglobulin or antibody or any functional fragment of an immunoglobulin molecule which binds to the target antigen and or combinations thereof. Examples of such functional entities include complete antibody molecules, antibody fragments, such as Fᵥ single chain Fᵥ (scfv), complementarity determining regions (CDRs), V_{L} (light chain variable region), V_{H} (heavy chain variable region), Fab, F(ab′)₂ and any combination of those or any other functional portion of an immunoglobulin peptide capable of binding to target antigen.

Antibodies exist, e.g., as intact immunoglobulins or as a number of well characterized fragments produced by digestion with various peptidases. Thus, for example, pepsin digests an antibody below the disulfide linkages in the hinge region to produce F(ab′)₂ a dirtier of Fab which itself is a light chain joined to V_{H}-C_{H1} by a disulfide bond. The F(ab′)₂ may be reduced under mild conditions to break the disulfide linkage in the hinge region, thereby converting the F(ab′)₂ dimer into an Fab₁ monomer. The Fab monomer is essentially an Fab with part of the hinge region (see Paul, 1993). While various antibody fragments are defined in terms of the digestion of an intact antibody, one of skill will appreciate that such fragments may be synthesized de novo either chemically or by utilizing recombinant DNA methodology. Thus, the term antibody as used herein also includes antibody fragments either produced by the modification of whole antibodies or those synthesized de novo using recombinant DNA methodologies.

An “antibody heavy chain” as used herein refers to the larger of the two types of polypeptide chains present in all antibody molecules.

An “antibody light chain” as used herein refers to the smaller of the two types of polypeptide chains present in all antibody molecules.

The term “single chain antibody” refers to an antibody wherein the genetic information encoding the functional fragments of the antibody are located in a single contiguous length of DNA. For a thorough description of single chain antibodies, see Bird et al., 1988; Huston et al., 1988).

By “small interfering RNA” (siRNA) is meant, inter alia, an isolated dsRNA molecule comprised of both a sense and an anti-sense strand. In some embodiments, it is greater than 10 nucleotides in length. siRNA also refers to a single transcript which has both the sense and complementary antisense sequences from the target gene, e.g., a hairpin. siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides. RNA interference is a commonly used method to regulate gene expression. This effect is often achieved by using small interfering RNA, short hairpin RNA (shRNA), or a dicer substrate siRNA (DsiRNA) named for its complementary enzyme.

The term “humanized” refers to an antibody wherein the constant regions have at least about 80% or greater homology to human immunoglobulin. Additionally, sonic of the nonhuman, such as murine, variable region amino acid residues can be modified to contain amino acid residues of human origin. Humanized antibodies have been referred to as “reshaped” antibodies. Manipulation of the complementarity-determining regions (CDR) is a way of achieving humanized antibodies. See for example, Jones et al., 1986; Riechmann et al.. 1988, both of which are incorporated by reference herein. For a review article concerning humanized antibodies, see Winter & Milstein, 1991, incorporated by reference herein. See also U.S. Pat. Nos. 4,816,567; 5,482,856; 6,479,284; 6,677,436; 7,060,808; 7,906,625; 8,398,980; 8,436,150; 8,796,439; and 10,253,111; and U.S. Patent Application Publication Nos. 2003/0017534, 2018/0298087, 2018/0312588, 2018/0346564, and 2019/0151448, each of which is incorporated by reference in its entirety.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response. This immune response may involve either antibody production, or the activation of specific immunologically competent cells, or both. An antigen can be derived from organisms, subunits of proteinslantigens, killed or inactivated whole cells or lysates.

As used herein, the term “antisense oligonucleotide” or anti sense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the presently disclosed subject matter include, but are not limited to, phosphorothioate oligonucleotides and other modifications of oligonucleotides.

An “aptamer” is a compound that is selected in vitro to bind preferentially to another compound (for example, the identified proteins herein). Often, aptamers are nucleic acids or peptides because random sequences can be readily generated from nucleotides or amino acids (both naturally occurring or synthetically made) in large numbers but of course they need not be limited to these.

The term “aqueous solution” as used herein can include other ingredients commonly used, such as sodium bicarbonate described herein, and further includes any acid or base solution used to adjust the pH of the aqueous solution while solubilizing a peptide.

The term “binding” refers to the adherence of molecules to one another, such as, but not limited to. enzymes to substrates, ligands to receptors, antibodies to antigens, DNA binding domains of proteins to DNA, and DNA or RNA strands to complementary strands.

“Binding partner” as used herein refers to a molecule capable of binding to another molecule, The term “biocompatible” as used herein refers to a material that does not elicit a substantial detrimental response in the host.

As used herein, the terms “biologically active fragment” and “bioactive fragment” of a peptide encompass natural and synthetic portions of a longer peptide or protein that are capable of specific binding to their natural ligand and/or of performing a desired function of a protein, for example, a fragment of a protein of larger peptide which still contains the epitope of interest and is immunogenic.

The term “biological sample” as used herein refers to samples obtained from a subject, including but not limited to skin, hair, tissue, blood, plasma, cells, sweat, and urine.

As used herein, the term “chemically conjugated”, or “conjugating chemically” refers to linking the antigen to the carrier molecule. This linking can occur on the genetic level using recombinant technology, wherein a hybrid protein may be produced containing the amino acid sequences, or portions thereof, of both the antigen and the carrier molecule. This hybrid protein is produced by an oligonucleotide sequence encoding both the antigen and the carrier molecule, or portions thereof. This linking also includes covalent bonds created between the antigen and the carrier protein using other chemical reactions, such as, but not limited to reactions as described herein. Covalent bonds may also be created using a third molecule bridging the antigen to the carrier molecule. These cross-linkers are able to react with groups, such as but not limited to, primary amines, sulthydryls, carbonyls, carbohydrates, or carboxylic acids, on the antigen and the carrier molecule. Chemical conjugation also includes non-covalent linkage between the antigen and the carrier molecule.

A “coding region” of a gene comprises the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein refers to the broad concept of subunit sequence complementarity between two nucleic acids (e.g., two DNA molecules). When a nucleotide position in both of the molecules is occupied by nucleotides normally capable of base pairing with each other at a given position, the nucleic acids are considered to be complementary to each other at this position. Thus, two nucleic acids are complementary to each other when a substantial number (in some embodiments at least 50%) of corresponding positions in each of the molecules are occupied by nucleotides that can base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. By way of example and not limitation, the first region comprises a first portion and the second region comprises a second portion, whereby, when the first and second portions are arranged in an antiparallel fashion, in some embodiments at least about 50%, in some embodiments at least about 75%, in some embodiments at least about 90%, and in some embodiments at least about 95% of the nucleotide residues of the first portion are capable of base pairing with nucleotide residues in the second portion. In some embodiments, all nucleotide residues of the first portion are capable of base pairing with nucleotide residues in the second portion.

A “compound” as used herein refers to a polypeptide, an isolated nucleic acid, or other agent used in the method of the presently disclosed subject matter.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a condition, disease, or disorder for which the test is being performed.

A “test” cell is a cell being examined.

As used herein, the term “conservative amino acid substitution” is defined herein as an amino acid exchange within one of the five groups summarized in the following Table:

### Exemplary Conservative Amino Acid Substitutions

| Group | Characteristics | Amino Acids |
|---|---|---|
| A. | Small aliphatic, nonpolar, or | Ala, Ser, Thr, Pro, Gly |
| | slightly polar residues | |
| B. | Polar, negatively charged residues | Asp, Asn, Glu, Gln |
| | and their amides | |
| C. | Polar, positively charged residues | His, Arg, Lys |
| D. | Large, aliphatic, nonpolar residues | Met Leu, Ile, Val, Cys |
| E. | Large, aromatic residues | Phe, Tyr, Trp |

A “pathoindicative” cell is a cell that, when present in a tissue, is an indication that the animal in which the tissue is located (or from which the tissue was obtained) is afflicted with a condition, disease, or disorder.

A “pathogenic” cell is a cell that, when present in a tissue, causes, or contributes to a condition, disease, or disorder in the animal in which the tissue is located (or from which the tissue was obtained).

A tissue “normally comprises” a cell if one or more of the cell are present in the tissue in an animal not afflicted with a condition, disease, or disorder.

As used herein, the terms “condition”, “disease condition”, “disease”, “disease state”. and “disorder” refer to physiological states in which diseased cells or cells of interest can be targeted with the compositions of the presently disclosed subject matter. In some embodiments, a disease is cancer, which in some embodiments comprises a solid tumor.

As used herein, the term “diagnosis” refers to detecting a risk or propensity to a condition, disease, or disorder. In any method of diagnosis exist false positives and false negatives. Any one method of diagnosis does not provide 100% accuracy.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” or “therapeutically effective amount” refers to an amount of a compound or composition sufficient to produce a selected effect, such as but not limited to alleviating symptoms of a condition, disease, or disorder. In the context of administering compounds in the form of a combination, such as multiple compounds, the amount of each compound, when administered in combination with one or more other compounds, may be different from when that compound is administered alone. Thus, an effective amount of a combination of compounds refers collectively to the combination as a whole, although the actual amounts of each compound may vary. The term “more effective” means that the selected effect occurs to a greater extent by one treatment relative to the second treatment to which it is being compared.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA, and mRNA) or a defined sequence of amino acids and the biological properties resulting therefrom. Thus, a gene encodes a protein if transcription and translation of an mRNA corresponding to or derived from that gene produces the protein in a cell or other biological system and/or an in vitro or ex vivo system. Both the coding strand, the nucleotide sequence of which is identical to the mRNA sequence (with the exception of uracil bases presented in the latter) and is usually provided in Sequence Listing, and the non-coding strand, used as the template for transcription of a gene or cDNA, can be referred to as encoding the protein or other product of that gene or cDNA.

The term “epitope” as used herein is defined as small chemical groups on the antigen molecule that can elicit and react with an antibody. An antigen can have one or more epitopes. Most antigens have many epitopes: i.e., they are multivalent. In general, an epitope is roughly five amino acids or sugars in size. One skilled in the art understands that generally the overall three-dimensional structure, rather than the specific linear sequence of the molecule, is the main criterion of antigenic specificity.

As used herein, an “essentially pure” preparation of a particular protein or peptide is a preparation wherein in some embodiments at least about 95% and in some embodiments at least about 99%, by weight, of the protein or peptide in the preparation is the particular protein or peptide.

A “fragment”, “segment”, or “subsequence” is a portion of an amino acid sequence, comprising at least one amino acid, or a portion of a nucleic acid sequence comprising at least one nucleotide. The terms “fragment”, “segment”, and “subsequence” are used interchangeably herein.

As used herein, the term “fragment”, as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be at least about 20 nucleotides in length, typically, at least about 50 nucleotides, more typically, from about 50 to about 100 nucleotides, in some embodiments, at least about 100 to about 200 nucleotides, in some embodiments, at least about 200 nucleotides to about 300 nucleotides, yet in some embodiments, at least about 300 to about 350, in some embodiments, at least about 350 nucleotides to about 500 nucleotides, yet in some embodiments, at least about 500 to about 600, in some embodiments, at least about 600 nucleotides to about 620 nucleotides, yet in some embodiments, at least about 620 to about 650, and most in some embodiments, the nucleic acid fragment will be greater than about 650 nucleotides in length. In the case of a shorter sequence, fragments are shorter,

As used herein, a “functional” biological molecule is a biological molecule in a form in which it exhibits a property by which it can be characterized. A functional enzyme, for example, is one that exhibits the characteristic catalytic activity by which the enzyme can be characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′-ATTGCC-5′ and 3′-TATGGC-5′ share 50% homology.

As used herein, “homology” is used synonymously with “identity”.

The determination of percent identity between two nucleotide or amino acid sequences can be accomplished using a mathematical algorithm. For example, a mathematical algorithm useful for comparing two sequences is the algorithm of Karlin & Altschul, 1990a, modified as in Karlin & Altschul, 1993). This algorithm is incorporated into the NBLAST and XBLAST programs of Altschul et al., 1990a, and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al., 1997. Alternatively, PSI-Blast or PHI-Blast can be used to perform an iterated search which detects distant relationships between molecules (Altschul et al., 1997) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

The tern “ingredient” refers to any compound, whether of chemical or biological origin, that can be used in cell culture media to maintain or promote the proliferation, survival, or differentiation of cells.

The terms “component”, “nutrient”, “supplement”, and ingredient” can be used interchangeably and are all meant to refer to such compounds. Typical non-limiting ingredients that are used in cell culture media include amino acids, salts, metals, sugars, lipids, nucleic acids, hormones, vitamins, fatty acids, proteins, and the like. Other ingredients that promote or maintain cultivation of cells ex vivo can be selected by those of skill in the art, in accordance with the particular need.

As used herein “injecting”, “applying”, and administering” include administration of a compound of the presently disclosed subject matter by any number of routes and modes including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, vaginal, and rectal approaches.

Used interchangeably herein are the terms: 1) “isolate” and “select”; and 2) “detect” and “identify”.

The term “isolated”, when used in reference to compositions and cells, refers to a particular composition or cell of interest, or population of cells of interest, at least partially isolated from other cell types or other cellular material with which it naturally occurs in the tissue of origin. A composition or cell sample is “substantially pure” when it is at least 60%, or at least 75%, or at least 90%, and, in certain cases, at least 99% free of materials, compositions, cells other than composition or cells of interest. Purity can be measured by any appropriate method, for example, by fluorescence-activated cell sorting (FACS), or other assays which distinguish cell types. Representative isolation techniques are disclosed herein for antibodies and fragments thereof.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, a “ligand” is a compound that specifically or selectively binds to a target compound. A ligand (e.g., an antibody) “specifically binds to”, “is specifically immunoreactive with”, “having a selective binding activity”, “selectively binds to” or “is selectively immunoreactive with” a compound when the ligand functions in a binding reaction which is determinative of the presence of the compound in a sample of heterogeneous compounds. Thus, under designated assay (e.g., immunoassay) conditions, the ligand binds preferentially to a particular compound and does not bind to a significant extent to other compounds present in the sample. For example, an antibody specifically or selectively binds under immunoassay conditions to an antigen bearing an epitope against which the antibody was raised. A variety of immunoassay formats may be used to select antibodies specifically immunoreactive with a particular antigen. For example, solid-phase ELISA immunoassays are routinely used to select monoclonal antibodies specifically immunoreactive with an antigen, See Harlow & Lane, 1988, for a description of immunoassay formats and conditions that can be used to determine specific immunoreactivity.

A “receptor” is a compound that specifically or selectively binds to a ligand. A ligand or a receptor (e.g., an antibody) “specifically binds to”, “is specifically immunoreactive with”, “having a selective binding activity”, “selectively binds to” or “is selectively immunoreactive with” a compound when the ligand or receptor functions in a binding reaction which is determinative of the presence of the compound in a sample of heterogeneous compounds. Thus, under designated assay (e.g., immunoassay) conditions, the ligand or receptor binds preferentially to a particular compound and does not bind in a significant amount to other compounds present in the sample. For example, a polynucleotide specifically or selectively binds under hybridization conditions to a compound polynucleotide comprising a complementary sequence; an antibody specifically or selectively binds under immunoassay conditions to an antigen bearing an epitope against which the antibody was raised. A variety of immunoassay formats may be used to select antibodies specifically immunoreactive with a particular protein. For example, solid-phase ELBA immunoassays are routinely used to select monoclonal antibodies specifically immunoreactive with a protein. See Harlow & Lane, 1988 for a description of immunoassay formats and conditions that can be used to determine specific or selective immunoreactivity.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, such as but not limited to through ionic or hydrogen bonds or van der Waals interactions.

The terms “measuring the level of expression” and “determining the level of expression” as used herein refer to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

The term “modulate” as used herein refers to changing the level of an activity, function, or process. The tern “modulate” encompasses both inhibiting and stimulating an activity, function, or process. The term “modulate” is used interchangeably with the term “regulate” herein.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil).

As used herein, the term “nucleic acid” encompasses RNA as well as single and double-stranded DNA and cDNA. Furthermore, the terms, “nucleic acid”, “DNA”, “RNA” and similar terms also include nucleic acid analogs, i.e. analogs having other than a phosphodiester backbone. For example, the so-called “peptide nucleic acids”, which are known in the art and have peptide bonds instead of phosphodiester bonds in the backbone, are considered within the scope of the presently disclosed subject matter. By “nucleic acid” is meant any nucleic acid, whether composed of deoxyribonucleosides or ribonucleosides, and whether composed of phosphodiester linkages or modified linkages such as phosphodiester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences”.

The term “nucleic acid construct” as used herein encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, G, C) in which “U” replaces “T”.

The term “otherwise identical sample” as used herein refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample may of course be a standard sample. By analogy, the term “otherwise identical” can also be used regarding regions or tissues in a subject or in an unaffected subject.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “peptide” typically refers to short polypeptides.

The term “pharmaceutical composition” refers to a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

“Pharmaceutically acceptable” means physiologically tolerable, for either human or veterinary application. Similarly, “pharmaceutical compositions” include formulations for human and veterinary use.

As used herein, the term “pharmaceutically acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Plurality” means at least two.

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” refers to non-naturally occurring peptides or polypeptides. Synthetic peptides or polypeptides can be synthesized, for example, using an automated polypeptide synthesizer. Various solid phase peptide synthesis methods are known to those of skill in the art.

The term “prevent” as used herein means to stop something from happening, or taking advance measures against something possible or probable from happening. In the context of medicine, “prevention” generally refers to action taken to decrease the chance of getting a disease or condition. It is noted that “prevention” need not be absolute, and thus can occur as a matter of degree.

A “preventive” or “prophylactic” treatment is a treatment administered to a subject who does not exhibit signs, or exhibits only early signs, of a condition, disease, or disorder. A prophylactic or preventative treatment is administered for the purpose of decreasing the risk of developing pathology associated with developing the condition, disease, or disorder.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross & Mienhofer, 1981 for suitable protecting groups.

As used herein. “protecting group” with respect to a terminal carboxy group refers to a terminal carboxyl group of a peptide, which terminal carboxyl group is coupled with any of various carboxyl-terminal protecting groups. Such protecting groups include, for example, tent-butyl, benzyl, or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus, the right-hand end of a polypeptide sequence is the carboxyl-terminus.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process.

A “highly purified” compound as used herein refers to a compound that is in some embodiments greater than 90% pure. that is in sonic embodiments greater than 95% pure, and that is in some embodiments greater than 98% pure.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function promoter, origin of replication, ribosome-binding site, etc.) as well.

A host cell that comprises a recombinant polynucleotide is referred to as a “recombinant host cell”. A gene which is expressed in a recombinant host cell wherein the gene comprises a recombinant polynucleotide, produces a “recombinant polypeptide”.

A “recombinant polypeptide” is one which is produced upon expression of a recombinant polynucleotide.

The term “regulate” refers to either stimulating or inhibiting a function or activity of interest.

As used herein, term “regulatory elements” is used interchangeably with “regulatory sequences” and refers to promoters, enhancers, and other expression control elements, or any combination of such elements.

As used herein, the term “secondary antibody” refers to an antibody that binds to the constant region of another antibody (the primary antibody).

As used herein, the term “single chain variable fragment” (scFv) refers to a single chain antibody fragment comprised of a heavy and light chain linked by a peptide linker. In some cases, scFv are expressed on the surface of an engineered cell, for the purpose of selecting particular scFv that bind to an antigen of interest.

As used herein, the term “mammal” refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

The term “subject” as used herein refers to a member of species for which treatment and/or prevention of a disease or disorder using the compositions and methods of the presently disclosed subject matter might be desirable, Accordingly, the term “subject” is intended to encompass in some embodiments any member of the Kingdom Animalia including, but not limited to the phylum Chordata (e.g., members of Classes Osteichthyes (bony fish), Amphibia (amphibians), Reptilia (reptiles), Ayes (birds), and Mammalia (mammals), and all Orders and Families encompassed therein.

The compositions and methods of the presently disclosed subject matter are particularly useful for warm-blooded vertebrates, Thus, in some embodiments the presently disclosed subject matter concerns mammals and birds. More particularly provided are compositions and methods derived from and/or for use in mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the use of the disclosed methods and compositions on livestock, including but not limited to domesticated swine (pigs and hogs), ruminants, horses, poultry, and the like.

As used herein, “substantially homologous amino acid sequences” includes those amino acid sequences which have at least about 95% homology, in some embodiments at least about 96% homology, more in some embodiments at least about 97% homology, in some embodiments at least about 98% homology, and most in some embodiments at least about 99% or more homology to an amino acid sequence of a reference antibody chain. Amino acid sequence similarity or identity can be computed by using the BLASTP and TBLASTN programs which employ the BLAST (basic local alignment search tool) 2.0,14 algorithm. The default settings used for these programs are suitable for identifying substantially similar amino acid sequences for purposes of the presently disclosed subject matter.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. In some embodiments, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 99% or more. Substantial identity of nucleic acid sequences can be determined by comparing the sequence identity of two sequences, for example by physical/chemical. methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO₄, 1 mM EDTA at 50° C. with washing in 2× standard saline citrate (SSC). 0.1% SDS at 50° C.; in some embodiments in 7% (SDS), 0.5 M NaPO₄, 1 mM EDTA 50° C. with washing in 1× SSC, 0.1% SDS at 50° C.; in some embodiments 7% SDS, 0.5 M NaPO₄, 1 mM EDTA at 50° C. with washing in 0.5× SSC, 0.1% SDS at 50° C.; and more in some embodiments in 7% SDS, 0.5 M NaPO₄, 1 mM EDTA at 50° C. with washing in 0.1×SSC. 0.1% SDS at 65° C. Suitable computer algorithms to determine substantial similarity between two nucleic acid sequences include. GCS program package (Devereux et al., 1984), and the BLASTN or FASTA programs (Altschul et al., 1990a; Altschul et al., 1990b; Altschul et al., 1997). The default settings provided with these programs are suitable for determining substantial similarity of nucleic acid sequences for purposes of the presently disclosed subject matter.

A “sample” as used herein refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

The term “standard” as used herein refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal. Such animals include mammals, in some embodiments, humans,

As used herein, a “subject in need thereof” is a patient, animal, mammal, or human, who will benefit from the method of this presently disclosed subject matter.

The term “substantially pure” describes a compound, e.g., a protein or polypeptide, which has been separated from components which naturally accompany it. Typically, a compound is substantially pure when in some embodiments at least 10%, in some embodiments at least 20%, in some embodiments at least 50%, in sonic embodiments at least 60%, in some embodiments at least 75%, in some embodiments at least 90%, and in some embodiments at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis, A compound, e.g., a protein, is also substantially purified when it is essentially free of naturally associated components or when it is separated from the native contaminants which accompany it in its natural state.

The term “symptom” as used herein refers to any morbid phenomenon or departure from the normal in structure, function, or sensation, experienced by the patient and indicative of disease. in contrast, a “sign” is objective evidence of disease. For example, a bloody nose is a sign. It is evident to the patient, doctor, nurse, and other observers.

A “therapeutic” treatment is a treatment administered to a subject who exhibits signs of pathology for the purpose of diminishing or eliminating those signs.

As used herein, the phrase “therapeutic agent” refers to an agent that is used to, for example, treat, inhibit, prevent, mitigate the effects of, reduce the severity of, reduce the likelihood of developing, slow the progression of, and/or cure, a disease or disorder.

The terms “treatment” and “treating” as used herein refer to both therapeutic treatment and prophylactic or preventative measures, wherein the object is to prevent or slow down (lessen) the targeted pathologic condition, prevent the pathologic condition, pursue or obtain beneficial results, and/or lower the chances of the individual developing a condition, disease, or disorder, even if the treatment is ultimately unsuccessful. Those in need of treatment include those already with the condition as well as those prone to have or predisposed to having a condition, disease, or disorder, or those in whom the condition is to be prevented.

As used herein, the terms “vector”, “cloning vector”, and “expression vector” refer to a vehicle by which a polynucleotide sequence (e.g., a foreign gene) can be introduced into a host cell, so as to transduce and/or transform the host cell in order to promote expression (e.g., transcription and translation) of the introduced sequence. Vectors include plasmids, phages, viruses, etc.

All genes, gene names, and gene products disclosed herein are intended to correspond to homologs and/or orthologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates.

### II. Exemplary Compositions of the Presently Disclosed Subject Matter

### II.A. Generally

Recently, there has been an increase in the number of oligonucleotide therapies, which alter specific gene and protein expression, for various cancers, cardiovascular conditions, neural disease, inflammatory diseases, and more. Currently, there are several platform technologies approved to deliver nucleic acid therapeutics, including chemically modified antisense oligonucleotides, GalNAc-conjugated siRNA, adeno-associated virus AAV, and lipid nanoparticles (LNPs). While these therapies are gaining more popularity, there are still many hurdles to producing them, as they rapidly degrade in vivo, particularly for unmodified nucleic acid based drugs.

A key challenge in the development of therapeutic application of molecular medicines (siRNA, mRNAs, miRNAs) is an efficient intracellular delivery to target tissues by a non-viral carrier. While LNPs for molecular-based therapeutics have been designed for depot delivery (mRNA vaccines), development has lagged for targeted systemic delivery. This clearly suggests that there is a critical need for the development of nanocarrier especially, ligand-conjugated nanocarriers, including LNPs, for a targeted delivery of RNA therapeutics.

Several siRNA moieties are directly linked to target motifs for organ or cellular-based delivery. Inclisiran ((Am-sp-(2′-deoxy-2′-fluoro)C-sp-Am-(2′-deoxy-2′-fluoro)A-(2′-deoxy-2′-fluoro)A-(2′-deoxy-2′-fluoro)A-Gm-(2′-deoxy-2′-fiuoro)C-Am-(2′-deoxy-2′-fiuoro)A-Am-(2′-deoxy-2′-fluoro)A-Cm-(2′-deoxy-2′-fluoro)A-Gm-(2′-deoxy-2′-fluoro)G-Um-(2′-deoxy-2′-fluoro)C-Um-Am-Gm-sp-Am-sp-Am), complex with RNA (Cm-sp-Um-sp-Am-Gm-Am-Cm-(2′-deoxy-2′-fluoro)C-Um-(2′-deoxy-2′-fluoro)G-Um-dT-Um-Um-Gm-Cm-Um-Um-Um-Um-Gm-Um) 3′-(((2S,4R)-1-(29-((2-(acetylamino)-2-deoxy-beta-D-galactopyranosyl)oxy)-14,14-bis((3-((3-((5-((2-(acetylamino)-2-deoxy-beta-D-galactopyranosyl)oxy)-1-oxopentyl)amino)propyl)amino)-3-oxopropoxy)methyl)-1,2,19,25-tetraoxo-16-oxa-13,20,24-triazanonacos-1-yl)-4-hydroxy-2-pyrrolidinyl)methyl hydrogen phosphate)), a recent FDA-approved molecular-based siRNA drug, was bioconjugated with a clustered GalNAC moiety. GalNAC specifically binds to asialo-glycoprotein receptors on hepatocytes and specifically delivers the siRNA into liver cells. The bioconjugated siRNA silences PCSK9 synthesis, specifically in hepatocytes, in a dose dependent manner, leading to a reduction in hypercholesteremia. Although many of these direct bioconjugated nucleic acid therapeutics induce significant long-lasting effects, they are limited because of high treatment costs (e.g., $300,000 to $2.1 million/year).

Thus, disclosed herein is a nanoparticle approach that has been engineered and validated to achieve selective liver targeting while “shielding” the immunogenic, toxic, and metabolizable siRNA. Specifically, a carbohydrate conjugated entrapped lipid nanoparticle has been designed for hepatocellular delivery that delivers siRNA. Galactosyl has been bioconjugated to cholesterol, and this targeted moiety has been utilized as a component of the LNP. As proof of concept, the bioactive siRNA component of Inclisiran (i.e., lacking the GalNac moiety) has been encapsulated within the LNP, as the targeting motif is now on the outside of the LNP.

In some embodiments, the compositions of the presently disclosed subject matter comprise, consist essentially of, or consist of galactosyl conjugated lipid nanoparticles (LNPs), including but not limited to N-acetylgalactosamine (GalNAc) conjugated LNPs, which have one or more active agents encapsulated therein. In some embodiments, the galactosyl LNP further comprises a lipid component comprising about 5-10% cholesterol. In some embodiments, the galactosyl conjugated LNP comprises a lipid component comprising D-Lin-MC3-DMA, cholesterol, DSPC, and DMG-2000-PEG.

In some embodiments, the galactosyl conjugated LNPs have one or more galactosyl moieties bioconjugated to cholesterol moieties present with the lipid component of the galactosyl conjugated LNPs. Thus, in sonic embodiments the galactosyl moieties are exposed to the environment in which the galactosyl conjugated LNPs are present, meaning that the galactosyl moieties are external to the lipid component of the galactosyl conjugated LNPs such that they are available to interact with other molecules including but not limited to molecules on the surfaces of hepatocytes. As a result, in some embodiments the galactosyl conjugated LNPs can be used to target the active agents to target cells, tissues, and organs including but not limited to hepatocytes and/or the liver.

In some embodiments, the active agent is an inhibitory nucleic acid, optionally an siRNA, Any siRNA that targets any gene product (e.g., mRNA) for which down-regulation of a biological activity of the gene product might be of interest can be employed in the compositions and methods of the presently disclosed subject matter to target said inhibitory nucleic acid, optionally siRNA to target cells, tissues, and organs including but not limited to hepatocytes and/or the liver. By way of example and not limitation, in some embodiments the inhibitory nucleic acid inhibits a biological activity of a PCSK9 gene product, optionally a human PCSK9 gene product, and further optionally wherein the inhibitory nucleic acid is an inclisiran derivative lacking its GalNAc moiety. In some embodiments, the galactosyl conjugated LNP targets the active agent to a cell, tissue, or organ of interest, optionally wherein the cell is a hepatocyte and/or the organ is liver.

Using the equation below, the lipid concentration was back calculated to 0.75 mM.

[Lipid]=[siRNA]*FRR*NPX

where FRR=3, N/P=2*, X=Ionizable Component Mol Fraction=0.5, and [siRNA]=0.021 gLmol PO43-**333.8 g=0.063 mM

It was assumed that an interaction of one positive charge on the amine of the lipid molecule interacted with one PO₄ of the siRNA. The concentration of reconstituted siRNA was measured using a Cytation 3 instrument.

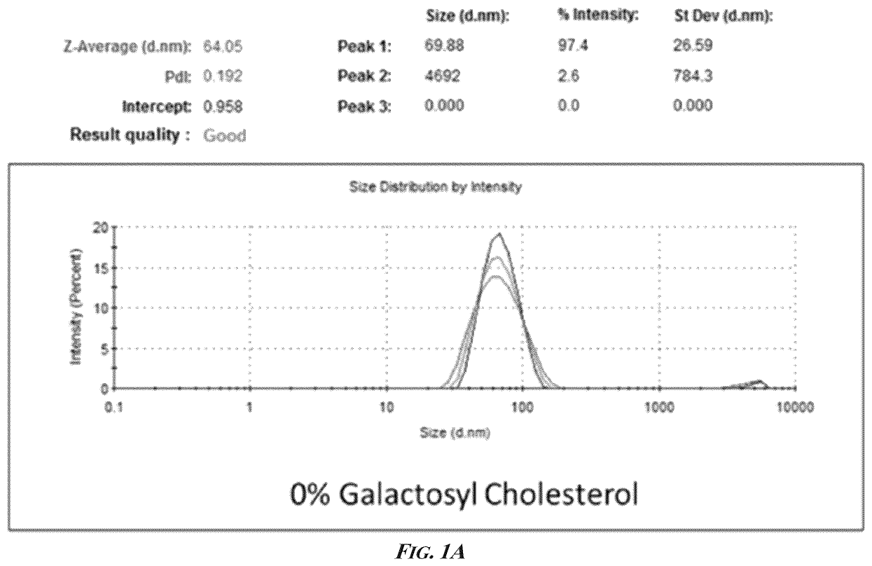

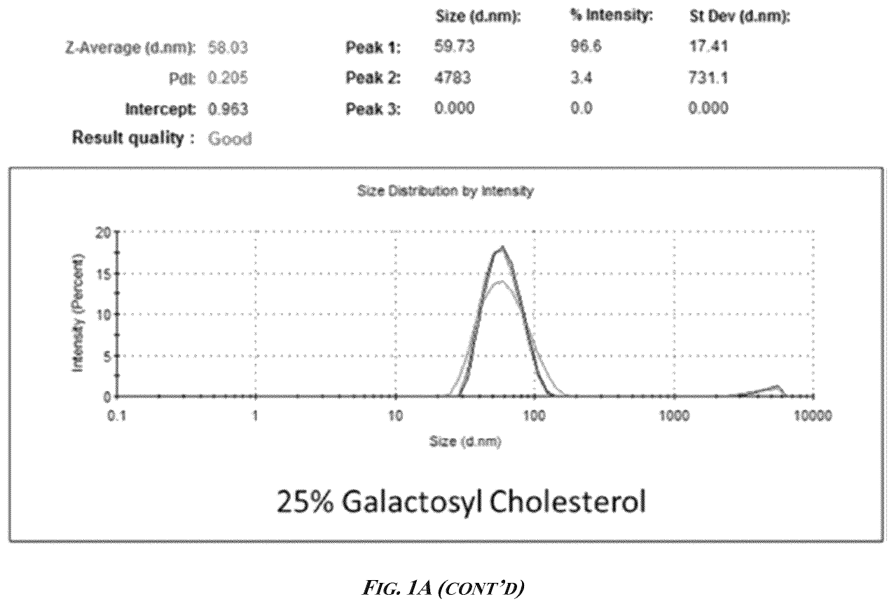

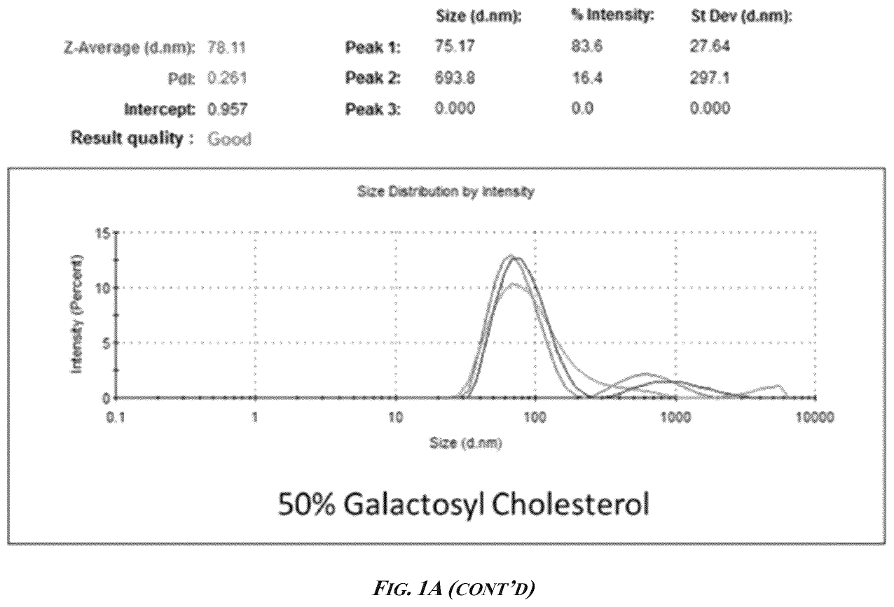

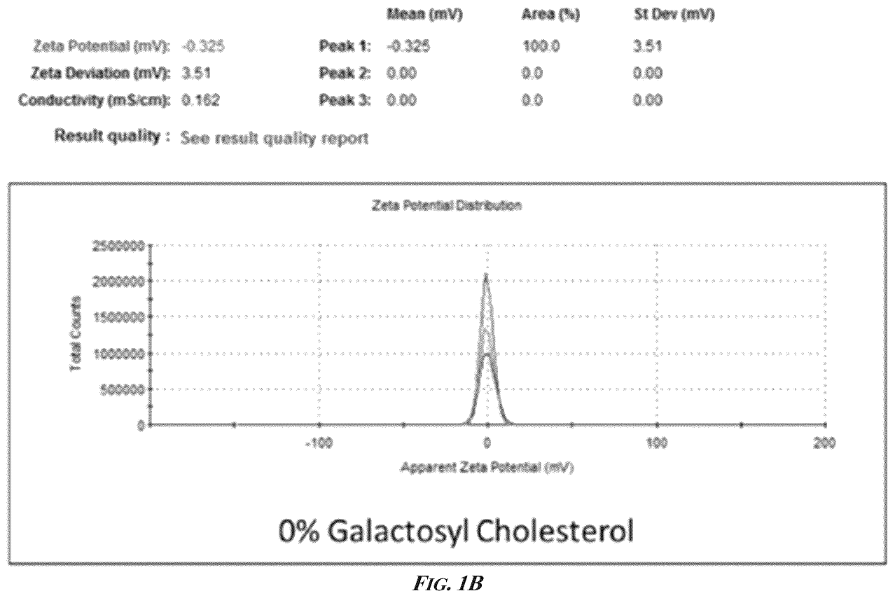

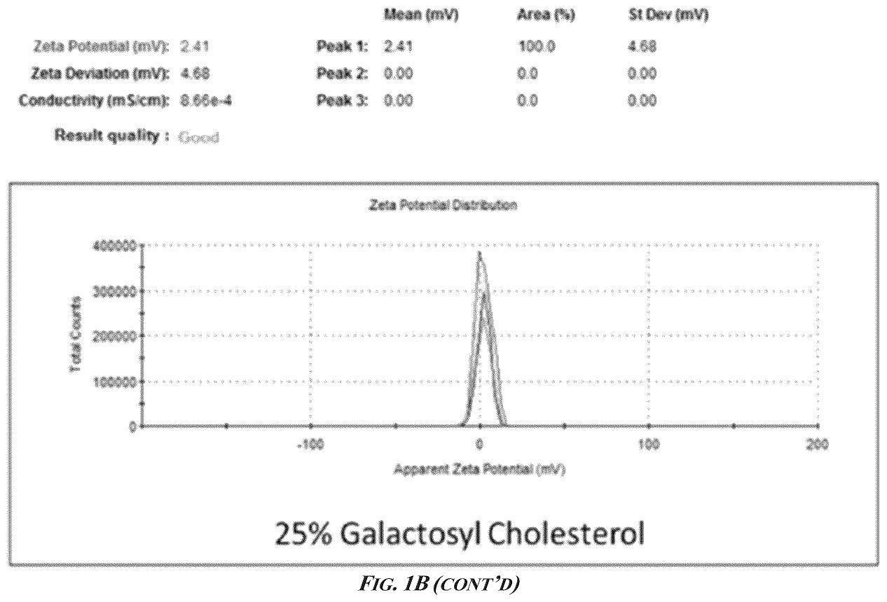

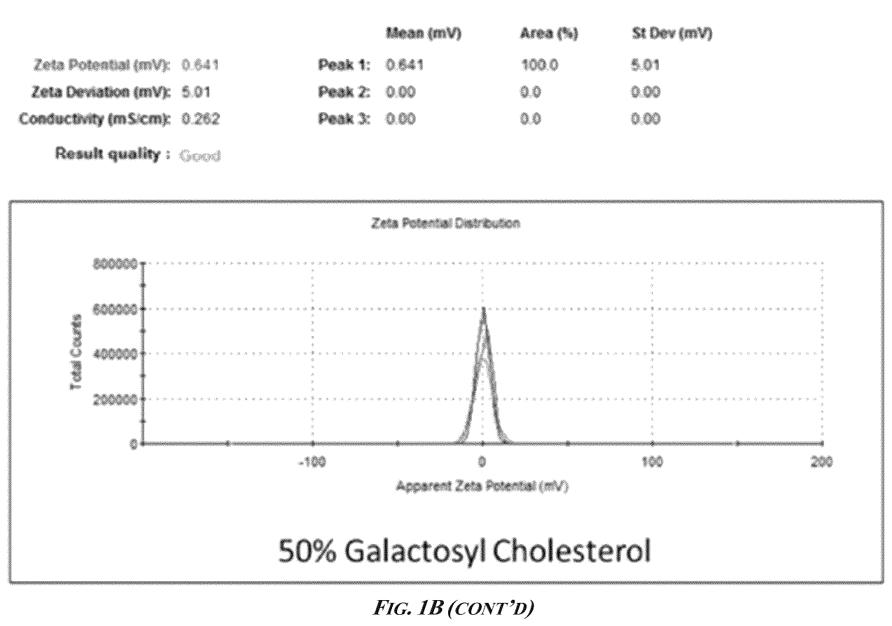

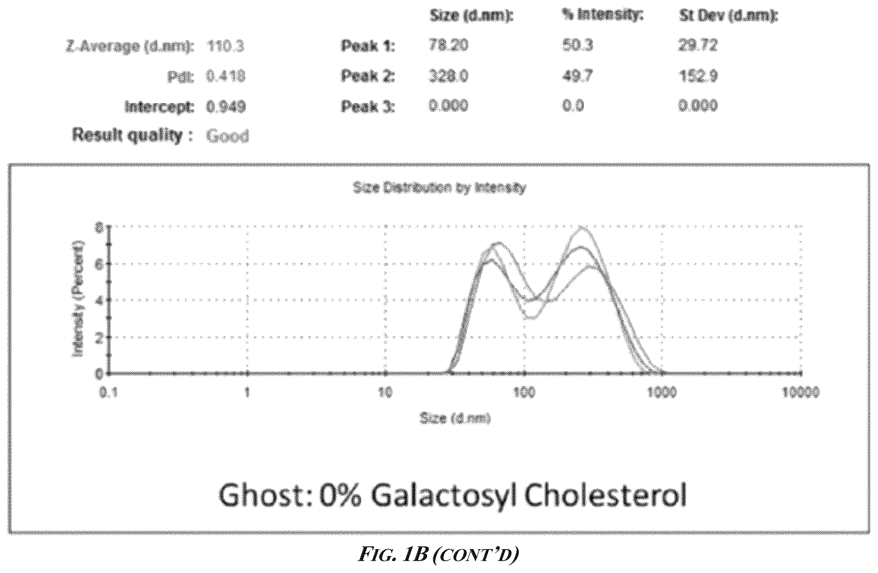

To achieve formulation of LNPs with a ILLS measurement of 100 nM, the aqueous siRNA and its organic ethanolic lipid solutions (molar ratios seen in Table 1) were combined via microfluidic mixing at a total flow rate of 12 mL/min, flow ratio of 3:1 aqueous:organic, total volume of 0.8 mL, starting waste volume of 0.25 mL, and ending waste volume of 0.05 mL. Post formulation processing of LNPs were characterized for size through dynamic light scattering using a Malvern Zetasizer (see FIG. 1) Particles were then dialyzed against PBS (pH 7.4) for 2 hours by injecting each sample into a Slide-A-Lyzer 0.1-0.5 mL dialysis cassette. The PBS dialyzed galactosyl-cholesterol LNPs that encapsulated siRNA achieved a narrow distribution with an approximate size of 90 nm.

The nitrogen to phosphorus (N/P) ratio is the molar ratio between the positively charged nitrogen and negatively charged nucleic acid backbone assuming one phosphorus per nitrogenous base and one nitrogen per ionizable (e.g., cationic) lipid. The respective charge difference helps form and stabilize the particle as well as encapsulate nucleic acids because they promote complexation between the nucleic acid backbone and cationic lipid head. There is an optimal ratio which has high stability, a high encapsulation efficacy, a narrow size distribution, and a size around 100 nm. The regular LNP has a N/P ratio between 2-8. Flow Rate Ratio (FRR), the relative proportions of aqueous and organic phase mixing, can be optimized to improve encapsulation efficiency and particle size. The proposed LNP formulations have an optimized ERR of 3:1. However, other FRRs, including but not limited to 1:3, 1;1, 2:1, and 1:2 can also be employed.

The nature and pH of the buffer changes the relative charge and solubility of lipids and nucleic acids thereby affecting LNP self-assembly by altering the interactions governing LNP self-assembly through changing constituent properties, allowing the control of LNP properties such as size, stability, zeta potential, and encapsulation efficiency. Thus far, three buffers have been considered; Citrate 1M pH 4.5, Phosphate Buffer 1M pH<5, and Sodium Acetate 0.1M pH 5 (Races et al., 2020).

In addition to the treatment of hypercholesterolemia, these LNPs are a platform for in vivo and in vitro gene expression manipulation. Using Gal-Chol, the presently disclosed LNPs can be used to treat various diseases affecting hepatocytes including hypercholesterolemia, hepatocytes with hexose sugar conjugation to asialoglycoprotein receptor 1 (ASGPR); hepatocellular carcinoma (HCC) including but not limited to HCC stages I-III; nonalcoholic fatty liver disease (NAFLD), nonalcoholic steatohepatitis NASH) ; hepatic cirrhosis; chronic active hepatitis, hepatitis resulting from infection with one or more of hepatitis viruses A-E, and hepatocellular hepatitis resulting from infection with Epstein Barr virus. In vitro LNPs can be used to knock down proteins for biological studies. In addition, as a therapy, LNP formulations can be optimized for in vivo delivery of RNAi to different tissues. This is especially useful in combination with other drugs to treat cancers as they quickly gain resistance to small molecule and immunogenic drugs, leading to disease relapse. Proposed therapies and rationales are listed below:

PCSK9. Inclisiran, a recently FDA-approved molecular-based (siRNA) drug, is bioconjugated with a clustered GalNAc moiety. GalNAc specifically binds to asialo-glycoprotein receptors (ASGPR) on hepatocytes and delivers the siRNA into liver cells. The bioconjugated siRNA silences PCSK9 synthesis in hepatocytes, in a dose dependent manner, leading to a drastic reduction in hypercholesterolemia. However, as stated there are many drawbacks to this direct conjugation approach, hence the development of the LNP based therapy (Lodish et al., 2021; Arnold & Koenig, 2022).

AID. AID overexpression can lead to genomic instability, resisting cell death, tumor-promoting inflammation; inducing angiogenesis, and enabling replicative immortality. As such, knocking down AID halts cancer cell proliferation through multiple pathways.

NRF2. Overactive NFR2 plays a role in many of the hallmarks of cancer including sustaining proliferative signaling, resisting cell death, evading growth suppressors, enabling replicative immortality; and inducing angiogenesis. Therefore, suppressing the expression of NRF2 helps kill cancer cells through multiple pathways (Gutschner & Diederichs, 2012; Sporn & Liby, 2012).

GAPDH. Because many cancer cells are dominated by the Warburg Effect (an increased reliance on glycolysis), knocking down GAPDH, a glycolytic master regulator which is frequently upregulated in cancers, offers a promising avenue to deregulate cancer cell energetics (Gutschner & Diederichs, 2012; Zhang et al., 2015).

p53. p53 loss of function leads to genomic instability, resisting cell death, tumor-promoting inflammation, enabling replicative immortality, and inducing angiogenesis. Therefore, it is a promising and widely researched target in developing cancer therapeutics (Gutschner & Diederichs, 2012; Lodish et al., 2021).

HDAC2. Histone Deacetylase 2 (HDAC2) is an oncogene which regulates the acetylation status of proteins to alter function independently and/or through partner binding. The most common of which is via deacetylation of histones. These modifications are key mediators of diseases, most notably cancer, as they alter chromatin accessibility and gene expression leading to aberrant activation/suppression of multiple pathways. As a result, the use of HDAC inhibitors has been an area of great investigation for the control of cancer, however, the development of specific inhibitors remains challenging due to the homology of disparate HDAC/SIRT proteins (Melesina et al 2021). Thus, we selected HDAC2, a key oncogenic target, to expand and validate our siRNA incorporating LNP technology past PCSK9 inhibition. FIDAC2 is a prime target as it is overexpressed and correlates with aggressiveness in a variety of cancers (breast, prostate, pancreatic and. HCC) and furthermore, inhibition of HDAC2 with siRNA has been shown to attenuate not only cell viability but also cellular responses to other common chemotherapeutic agents (Liu et al. 2021, Schuler et al 2010, Jo et al 2023). Additionally, HDAC2 is thought to play a role in many metabolic diseases of the liver including NAFLD (Liu et al. 2021), further extending the implications of our work. Thus, the ability to deliver siRNA targeting this molecular mediator is crucial in advancing single and combinatorial agents in cancer care.

### II.B. Pharmaceutical Compositions and Administration

The presently disclosed subject matter is also directed to methods of administering the compounds of the presently disclosed subject matter to a subject. Thus, in some embodiments of the presently disclosed subject matter, the compositions are pharmaceutical compositions, optionally pharmaceutical compositions that are pharmaceutically acceptable for use in mammals such as but not limited to humans.

Pharmaceutical compositions comprising the present compounds are administered to a subject in need thereof by any number of routes including, but not limited to, topical, oral, intravenous, intramuscular, intra-arterial, intramedullary, intrathecal, intraventricular, transdermal, subcutaneous, intraperitoneal, intranasal, enteral, topical, sublingual, or rectal means. As such, in some embodiments the presently disclosed compositions are administered by injecting the composition subcutaneously, intraperitoneally, into adipose tissue, and/or intramuscularly into the subject.

In accordance with one embodiment, a method for treating a subject in need of such treatment is provided. The method comprises administering a pharmaceutical composition comprising at least one compound of the presently disclosed subject matter to a subject in need thereof. Compounds identified by the methods of the presently disclosed subject matter can be administered with known compounds or other medications as well.

The pharmaceutical compositions useful for practicing the presently disclosed subject matter may be administered to deliver a dose of between 1 ng/kg/day and 100 mg/kg/day.

The presently disclosed subject matter encompasses the preparation and use of pharmaceutical compositions comprising a compound useful for treatment of the diseases and disorders disclosed herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “physiologically acceptable” ester or salt means an ester or salt form of the active ingredient which is compatible with any other ingredients of the pharmaceutical composition, which is not deleterious to the subject to which the composition is to be administered,

The compositions of the presently disclosed subject matter may comprise at least one active peptide, one or more acceptable carriers, and optionally other peptides or therapeutic agents.

For in vivo applications, the compositions of the presently disclosed subject matter may comprise a pharmaceutically acceptable salt. Suitable acids which are capable of forming such salts with the compounds of the presently disclosed subject matter include inorganic acids such as hydrochloric acid; hydrobromic acid, perchloric acid, nitric acid, thiocyanic acid, sulfuric acid, phosphoric acid and the like; and organic acids such as formic acid, acetic acid propionic acid, glycolic acid, lactic acid, anthranilic acid, cinnamic acid, naphthalene sulfonic acid, sulfanilic acid and the like.

Pharmaceutically acceptable carriers include physiologically tolerable or acceptable diluents, excipients, solvents, or adjuvants. The compositions are in some embodiments sterile and nonpyrogenic. Examples of suitable carriers include, but are not limited to, water, normal saline, dextrose, mannitol, lactose or other sugars, lecithin, albumin, sodium glutamate, cysteine hydrochloride, ethanol, polyols (propylene glycol, polyethylene glycol, glycerol, and the like), vegetable oils (such as olive oil), injectable organic esters such as ethyl oleate, ethoxylated isosteraryl alcohols, polyoxyethylene sorbitol and sorbitan esters, microcrystalline cellulose, aluminum methahydroxide, bentonite, kaolin, agar-agar and tragacanth, or mixtures of these substances, and the like.

In some embodiments wherein a composition of the presently disclosed subject matter is desired to induce an immune response, the compositions of the presently disclosed subject matter can further comprise an adjuvant. In some embodiments, the at least one adjuvant is selected from the group consisting of montanide ISA-51 (Seppic, Inc.), QS-21 (Aquila Pharmaceuticals, Inc.), tetanus helper peptides, GM-CSF, cyclophosamide, bacillus Calmette-Guerin (BCG), corynbacterium parvum, levamisole, azimezone, isoprinisone, dinitrochlorobenezene (DNCB), keyhole limpet hemocyanins Freunds adjuvant (complete and incomplete), mineral gels, aluminum hydroxide (Alum), lysolecithin, plutonic polyols, polyanions, peptides, oil emulsions, dinitrophenol, diphtheria toxin (DT).

The pharmaceutical compositions may also contain minor amounts of nontoxic auxiliary pharmaceutical substances or excipients and/or additives, such as wetting agents, emulsifying agents, pH buffering agents, antibacterial and antifungal agents (such as parabens, chlorobutanol, phenol, sorbin acid, and the like). Suitable additives include, but are not limited to, physiologically biocompatible buffers (e.g., tromethamine hydrochloride), additions (e.g., 0.01 to 10 mole percent) of chelants (such as, for example, DTPA or DTPA-bisamide) or calcium chelate complexes (as for example calcium DTPA or CaNaDTPA-hisamide), or, optionally, additions (e.g., 1 to 50 mole percent) of calcium or sodium salts (for example, calcium chloride, calcium ascorbate, calcium gluconate or calcium lactate). If desired, absorption enhancing or delaying agents (such as liposomes, aluminum monostearate, gelatin) may be used. The compositions can be prepared in conventional forms, either as liquid solutions or suspensions, solid forms suitable for solution or suspension in liquid prior to injection, or as emulsions. Pharmaceutical compositions according to the presently disclosed subject matter can be prepared in a manner fully within the skill of the art.

The compositions of the presently disclosed subject matter, pharmaceutically acceptable salts thereof, or pharmaceutical compositions comprising these compounds may be administered so that the compounds may have a physiological effect. Administration may occur enterally or parenterally; for example, orally, rectally, intracisternally, intravaginally, intraperitoneally, locally (e.g., with powders, ointments, or drops), or as a buccal or nasal spray or aerosol. Parenteral administration is preferred. Particularly preferred parenteral administration methods include intravascular administration (e.g., intravenous bolus injection, intravenous infusion, intra-arterial bolus injection, intra-arterial infusion, and catheter instillation into the vasculature), peri- and intra-target tissue injection, subcutaneous injection or deposition including subcutaneous infusion, intramuscular injection, and direct application to the target area, for example by a catheter or other placement device.

Where the administration of the peptide is by injection or direct application, the injection or direct application may be in a single dose or in multiple doses. Where the administration of the compound is by infusion, the infusion may be a single sustained dose over a prolonged period of time or multiple infusions.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

It will be understood by the skilled artisan that such pharmaceutical compositions are generally suitable for administration to animals of all sorts. Subjects to which administration of the pharmaceutical compositions of the presently disclosed subject matter is contemplated include, but are not limited to, humans and other primates, mammals including commercially relevant mammals such as cattle, pigs, horses, sheep, cats, and dogs, birds including commercially relevant birds such as chickens, ducks, geese, and turkeys.

A pharmaceutical composition of the presently disclosed subject matter may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the presently disclosed subject matter will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the presently disclosed subject matter may further comprise one or more additional pharmaceutically active agents. Particularly contemplated additional agents include anti-emetics and scavengers such as cyanide and cyanate scavengers.

Controlled- or sustained-release formulations of a pharmaceutical composition of the presently disclosed subject matter may be made using conventional technology.

As used herein, “additional ingredients” include, but are not limited to, one or more of the following: excipients; surface active agents; dispersing agents; inert diluents; granulating and disintegrating agents; binding agents; lubricating agents; sweetening agents, flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers, salts; thickening agents; fillers; emulsifying, agents; antioxidants; antibiotics; antifungal agents; stabilizing agents; and pharmaceutically acceptable polymeric or hydrophobic materials. Other “additional ingredients” which may be included in the pharmaceutical compositions of the presently disclosed subject matter are known in the art and described, for example in Gennaro, 1985; Gennaro, 1990; or Gennaro, 2003; each of which is incorporated herein by reference.

Typically, dosages of the compound of the presently disclosed subject matter which may be administered to an animal, in some embodiments a human, range in amount from 1 μg to about 100 g per kilogram of body weight of the animal. While the precise dosage administered will vary depending upon any number of factors, including but not limited to, the type of animal and type of disease state being treated, the age of the animal and the route of administration. In some embodiments, the dosage of the compound will vary from about 1 mg to about 10 g per kilogram of body weight of the animal. In another aspect, the dosage will vary from about 10 mg to about 1 g per kilogram of body weight of the animal.

The compound may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type of cancer being diagnosed, the type and severity of the condition or disease being treated, the type and age of the animal, etc.

Suitable preparations include injectables, either as liquid solutions or suspensions, however, solid forms suitable for solution in, suspension in, liquid prior to injection, may also be prepared. The preparation may also be emulsified, or the polypeptides encapsulated in liposomes. The active ingredients are often mixed with excipients which are pharmaceutically acceptable and compatible with the active ingredient. Suitable excipients are, for example, water saline, dextrose, glycerol, ethanol, or the like and combinations thereof. In addition, if desired, the vaccine preparation may also include minor amounts of auxiliary substances such as wetting or emulsifying agents, pH buffering agents, and/or adjuvants.

The presently disclosed subject matter also includes a kit comprising the composition of the presently disclosed subject matter and an instructional material which describes administering the composition to a subject. In some embodiments, this kit comprises a (in some embodiments sterile) solvent suitable for dissolving or suspending the composition of the presently disclosed subject matter prior to administering the compound to the subject.

As used herein, an “instructional material” includes a publication, a recording, a diagram, or any other medium of expression which can be used to communicate the usefulness of a composition of the presently disclosed subject matter in the kit for effecting alleviation of the various diseases or disorders recited herein. Optionally, or alternately, the instructional material may describe one or more methods of using the compositions for diagnostic or identification purposes or of alleviation the diseases or disorders in a cell or a tissue of a mammal. The instructional material of the kit of the presently disclosed subject matter may, for example, be affixed to a container which contains a composition of the presently disclosed subject matter or be shipped together with a container which contains the composition. Alternatively, the instructional material may be shipped separately from the container with the intention that the instructional material and the compound be used cooperatively by the recipient. The presently disclosed subject matter also related to methods for using the compositions of the presently disclosed subject matter for various purposes. For example, in some embodiments the presently disclosed subject matter also relates to methods for treating and/or preventing diseases, disorders, and/or conditions associated with inflammation.

### II.C. Dosages

An effective dose of a composition of the presently disclosed subject matter is administered to a subject in need thereof. A “treatment effective amount” or a “therapeutic amount” is an amount of a therapeutic composition sufficient to produce a measurable response (e.g., a biologically or clinically relevant response in a subject being treated, such as but not limited to a reduction in scarring and/or fibrosis, particularly as compared to the same subject had the subject not received the composition). Actual dosage levels of active ingredients in the compositions of the presently disclosed subject matter can be varied so as to administer an amount of the active compound(s) that is effective to achieve the desired therapeutic response for a particular subject. The selected dosage level will depend upon the activity of the composition, the route of administration, combination with other drugs or treatments, the severity of the disease, disorder, and/or condition being treated, and the condition and prior medical history of the subject being treated. However, it is within the skill of the art to start doses of the compositions of the presently disclosed subject matter at levels lower than required to achieve the desired therapeutic effect and to gradually increase the dosage until the desired effect is achieved, The potency of a composition can vary, and therefore a “treatment effective amount” can vary. However, using the methods described herein, one skilled in the art can readily assess the potency and efficacy of a composition of the presently disclosed subject matter and adjust the therapeutic regimen accordingly.

After review of the disclosure of the presently disclosed subject matter presented herein, one of ordinary skill in the art can tailor the dosages to an individual subject, taking into account the particular formulation, method of administration to be used with the composition, and particular disease, disorder, and/or condition treated. Further calculations of dose can consider subject height and weight, severity and stage of symptoms, and the presence of additional deleterious physical conditions. Such adjustments or variations, as well as evaluation of when and how to make such adjustments or variations, are well known to those of ordinary skill in the art of medicine.

In some embodiments, a pharmaceutically or therapeutically effective amount of a phototunable hydrogel of the presently disclosed subject matter is administered to a subject at a site of a wound and/or injury, and/or at a site where fibrosis is and/or might occur, and/or at a site where transition of fibroblasts to myofibroblasts would be undesirable.

### II.D. Routes of Administration

Suitable methods for administration of the compositions of the presently disclosed subject matter include, but are not limited to intravenous administration, oral delivery, and delivery directly to a target tissue or organ (e.g., a topical application and/or a site of injury such as but not limited to a muscle injury). Exemplary routes of administration include parenteral, enteral, intravenous, intraarterial, intracardiac, intrapericardial, intraosseal, intracutaneous, subcutaneous, intradermal, subdermal, transdermal, intrathecal, intramuscular, intraperitoneal, intrasternal, parenchymatous, oral, sublingual, buccal, inhalational, and intranasal. The selection of a particular route of administration can be made based at least in part on the nature of the formulation and the ultimate target site where the compositions of the presently disclosed subject matter are desired to act. In some embodiments, the method of administration encompasses features for regionalized delivery or accumulation of the compositions at the site in need of treatment. In some embodiments, the compositions are delivered directly into the site to be treated. By way of example and not limitation, in some embodiments a composition of the presently disclosed subject matter is administered to the subject via a route selected from the group consisting of intraperitoneal, intramuscular, intravenous, and intranasal, or any combination thereof.

The methods described herein use pharmaceutical compositions comprising the molecules described above, together with one or more pharmaceutically acceptable excipients or vehicles, and optionally other therapeutic and/or prophylactic ingredients. Such excipients include liquids such as water, saline, glycerol, polyethylene glycol, hyaluronic acid, ethanol, cyclodextrins, modified cyclodextrins sufobutyl ether cyclodextrins), etc. Suitable excipients for non-liquid formulations are also known to those of skill in the art. Pharmaceutically acceptable salts can be used in the compositions of the present invention and include, for example, mineral acid salts such as hydrochlorides, hydrobromides, phosphates, sulfates, and the like; and the salts of organic acids such as acetates, propionates, malonates, benzoates, and the like.

Additionally, auxiliary substances, such as wetting or emulsifying agents, biological buffering substances, surfactants, and the like, may be present in such vehicles. A biological buffer can be virtually any solution which is pharmacologically acceptable and which provides the formulation with the desired pH, i.e., a pH in the physiologically acceptable range. Examples of buffer solutions include saline, phosphate buffered saline, Tris buffered saline, Hank's buffered saline, and the like.

Depending on the intended mode of administration, the pharmaceutical compositions may be in the form of a liquid, suspension, cream, ointment, lotion, or the like, preferably in unit dosage form suitable for single administration of a precise dosage. The compositions can in some embodiments include one or more pharmaceutically acceptable carriers and, in addition, may include other pharmaceutical agents, adjuvants, diluents, buffers, etc.

In some embodiments, the mode of administration is a liquid form, which can then be cured by application of light of the appropriate wavelength, intensity, and duration to cure the phototunable hydrogels of the presently disclosed subject matter at a site of interest.

### III. Exemplary Methods for Using the Compositions of the Presently Disclosed Subject Matter

In some embodiments, the presently disclosed subject matter also relates to methods for treating and/or preventing diseases, disorders, and/or conditions associated with undesirable PCSK9 gene expression levels in cells, tissues, and/or organs using the compositions of the presently disclosed subject matter. In some embodiments, the presently disclosed methods comprise, consist essentially of, or consist of administering to a subject in need thereof an effective amount of a composition or mixture of a plurality of compositions as disclosed herein, wherein the presently disclosed compositions comprise, consist essentially of, or consist of one or more active agents that inhibit the activity of the gene with which the disease, disorder, or condition is associated. In some embodiments, the disease, disorder, and/or condition is hypercholesterolemia, an inflammatory disease, disorder, and; or condition including, but not limited to atherosclerosis, and/or sepsis, including consequences thereof such as but not limited to cardiovascular disease and its consequences and/or acute organ failure and its consequences.

By virtue of the exposed Galactosyl moiety, the presently disclosed LNPs can be used to deliver the active agents encapsulated therein to target tissues, Exemplary targets include hepatocytes. Other exemplary targets include the inflammasome and members thereof as well as cells involved with inflammatory processes in order to treat and/or prevent inflammatory diseases, disorders, and conditions, and cells and tissues of the cardiovascular system to treat and/or prevent cardiovascular diseases, disorders, and conditions. See e.g., Oikonomou & Antoniades, 2019; Mezu-Ndubuisi & Maheshwari, 2021.

As such, in some embodiments the presently disclosed subject matter relates to methods for targeting active agents to hepatocytes. In some embodiments, the methods comprise, consist essentially of, or consist of contacting hepatocytes with one or more compositions comprising one or more active agents, wherein the one or more active agents are encapsulated by a galactosyl conjugated lipid nanoparticle. (LNP). In some embodiments, the galactosyl LNP comprises a lipid component comprising D-Lin-MC3-DMA, cholesterol, DSPC, and DMG-2000-PEG, optionally wherein the cholesterol comprises one or more galactosyl moieties conjugated thereto, whereby the active agent is targeted to the hepatocyte.

In some embodiments, the disease, disorder, and/or condition to be treated with the compositions and methods of the presently disclosed subject matter is sepsis and septic shock. Septic shock is caused by an uncontrolled overwhelming acute immune response to bacterial infections and their byproducts in the blood causing a cytokine storm and subsequent organ failure. Decreasing hepatocyte secreted PCKS9 protein serum levels is associated with a decrease of septic shock onset probability and thus a greater chance for survival by helping to clear bacterial products and thus decreasing immune response. The presently disclosed PCSK9 siRNA-loaded hepatic-targeted LNP decreases relative concentrations of PCSK9 and PCSK9 mRNA. Thus, administration of these LNPs optionally in conjunction with antibiotics is thus a new treatment for sepsis and septic shock. While not wishing to be bound by any particular theory of operation, the presently disclosed PCSK9 siRNA loaded LNP gives the body a greater window to deal with the infection, and combined with antibiotics, the conventional yet underwhelming treatment for sepsis, addresses the infection at the cause and provides a greater capacity to clear the bacterial blood contaminants.

There are no current treatments for sepsis other than antibiotics. Inhibition of PCSK9 biological activities, optionally paired with antibiotics, can thus be used as a new treatment for sepsis. Walley and co-workers described PCSK9 biological activity as being important for the innate immune response and outcome in septic shock (Walley et al., 2014), and Boyd et al, found that overexpression of PCSK9 in plasma was associated with negative outcomes in sepsis, including reduced endotoxin clearance and the development of acute organ failure (Boyd et al., 2016). As set forth therein, LDLR helps clear bacterial lipids. The-se lipids are incorporated into cholesterol particles taken up by the PCSK9 levels are elevated in early sepsis. As PCSK9 levels surpass ˜220 ng/mL, organ failure and septic shock likelihood greatly increases as hepatic endotoxin uptake decreases (see FIG. 12).

It was further determined from a murine study that as compared to wild type mice, PCSK9 KO mice, upon LPS administration or induced sepsis, had reduced concentrations of TNF-alpha, IL-6, IL-10, and other pro-inflammatory cytokines. Organ failure is associated with overwhelming concentrations of these pro-inflammatory cytokines (so-called “cytokine storm”). PCSK9 KO mice had 55% lower endotoxin, subject to hepatic clearance.

Thus, in some embodiments a combination therapy is employed that drastically increases the chance of survival of septic shock caused by an uncontrolled overwhelming acute immune response to bacterial infections and their byproducts in the blood causing a cytokine storm and subsequent organ failure. Decreasing hepatocyte secreted PCKS9 protein serum levels is associated with a decrease of septic shock onset probability and thus a greater chance for survival by helping to clear bacterial products and thus decreasing immune response. The PCSK9 siRNA-loaded hepatic-targeted LNPs is disclosed herein to decrease relative concentrations of PCSK9 and PCSK9 mRNA. Accordingly, the presently disclosed LNPs optionally in combination with antibiotics could thus be employed for treating and/or preventing sepsis and septic shock.

In some embodiments, the disease, disorder, and/or condition to be treated with the compositions and methods of the presently disclosed subject matter is a cardiovascular disease. In some embodiments, the cardiovascular disease is atherosclerosis or a consequence of atherosclerosis. There are no conventional therapies for treating atherosclerosis, though FDA approved drugs can manage the symptoms. Likewise, the role of PCSK9 in atherosclerosis biology is currently unclear (see Wang et al, 2023). Even though there is evidence of PCSK9 being implicated in atherosclerotic progression, it has not been considered a target for therapeutic development. As previously stated in the sepsis rational, PCSK9 inhibition/degradation has been has demonstrated to drastically reduce LDL levels and to a lesser extent inflammation, both of which are key prognostic factors in developing atherosclerosis. Inducing NLRP3 and IL-6 mediated inflammation through oxidation, cholesterol particles build up in the bloodstream, residing in the intima where they are consumed by macrophages, which in turn develop into static foam cells. These foam cells continue to build up, resulting in atherosclerotic plaque and eventual bursting of the endothelial wall (see e.g., Lodish et al., 2021; Merćep et al., 2023; Momtazi-Borojeni et al., 2019). PCSK9 inhibition is hypothesized to reduce atherosclerotic plaque size and stability (Merćep et al., 2023), In this regard, it is postulated that if one can achieve tissue specific nanoparticle mediated subcellular delivery of siRNAs and SMDs targeting two discrete, converging pro-atherosclerotic pathways, such as the cholesterol metabolism (PCSK9) and the inflammasome (NLRP3), the co-delivery would be highly effective in treating inflammasome-linked cardiovascular diseases like coronary artery disease (CAD), myocardial infarction (MI), thrombosis, and atherosclerosis. Consequently, interrogation of the targeted delivery of PCSK9 siRNA therapeutics as disclosed herein and nanotherapeutics as disclosed herein can spearhead novel combinatorial drug combinations using preexisting drugs with improved efficacy due to the nanoparticle delivery strategy.

In some embodiments, atherosclerosis is targeted through multiple pathways. By way of example and not limitation, a combination therapy targeting two converging mechanisms can be employed. In some embodiments, the contributing metabolic pathways are treated by PCSK9 downregulation through the LNP system disclosed herein, and thus inhibiting pro-inflammatory pathways (e.g., with IL-6 or NLRP3 inhibitors) and/or coagulation pathways (e.g., with antiplatelet drugs) represent good targets to achieve synergy. Additionally, in some embodiments the pathways to target and the correct ratiometric doses can be screened against the predicted synergy score to find the desirable combinations (e.g., with the PCSK9 siRNA LNP disclosed herein) and relative doses that in some embodiments outperform the predictions. The traditional synergy screening model may not apply since the disease development implicates the function of multiple cell types. Thus, the synergy screening can be tested in a murine model or a complex novel cell culture model.

In some embodiments, the one or more active agents are inhibitory nucleic acids, optionally siRNAs. The inhibitory nucleic acid can be employed to inhibit biological activities of PCSK9 gene expression in hepatocytes. Any biological activity that is associated with PCSK9 gene expression can be targeted for inhibition using the methods of the presently disclosed subject matter. In some embodiments, the PCSK9 gene the expression of which is targeted is a human PCSK9 gene.

Thus, the methods of the presently disclosed subject matter can be employed to deliver inhibitory nucleic acids to cells, including but not limited to hepatocytes, tissues, and organs for which modulation of PCSK9 gene expression is desired. One exemplary inhibitor nucleic acid is inclisiran, which has been shown to inhibit the human PCSK9 gene to thereby treat hypercholesterolemia. As disclosed herein, the GalNAc moiety that is present on inclisiran can be removed when the inclisiran siRNA is encapsulated into a galactosyl LNP without negatively affecting the ability of the inclisiran derivative to target hepatocytes,

In some embodiments, hepatocyte-mediated PCSK9 expression is associated with other conditions besides hypercholesterolemia, hepatocytes with hexose sugar conjugation to asialoglycoprotein receptor I (ASGPR); hepatocellular carcinoma (HCC) including but not limited to HCC stages I-III; nonalcoholic fatty liver disease (NAELD), nonalcoholic steatohepatitis (NASH), hepatic cirrhosis, chronic active hepatitis, hepatitis resulting from infection with one or more of hepatitis viruses A-E, and hepatocellular hepatitis resulting from infection with Epstein Barr virus, and thus the presently disclosed LNPS, in some embodiments sugar-coated LNPs, can be employed to treat and/or prevent those diseases, disorders, and conditions. Exemplary non-hepatic diseases, disorders, and conditions that can be treated or prevented with the LNPs of the presently disclosed subject matter include, but are not limited to inflammatory diseases, disorders, and/or conditions including, but not limited to cardiovascular disease and its consequences including but not limited to atherosclerosis; sepsis, including consequences thereof such as but not limited to septic shock and cytokine storm; and/or acute organ failure and its consequences. In some embodiments, secretion of PCSK9 by hepatocytes, either alone or in combination with other cells, tissues, and organs, is associated with these conditions, and thus inhibiting PCSK9 biological activities using the compositions and methods of the presently disclosed subject matter can be used to treat and/or prevent these diseases, disorders, and/or conditions.

In some embodiments, the presently disclosed compositions and methods are designed to treat and/or prevent a cardiovascular disease (CVD), disorder, or condition selected from the group consisting of coronary artery disease (CAD), myocardial infarction, atherosclerosis, vascular thrombosis, and vascular inflammation. In some embodiments, an approach comprising targeting combinatorial pathways with nanotherapeutics in cardiovascular disease, especially atherosclerosis/MI, using biologics and small molecules is included within the presently disclosed subject matter.

Inflammation-related CVD is typically is associated with high degrees of fatality as current approaches to treatment are inadequate. Generally, symptoms are managed with standard medication. There are many cells implicated in the breadth of these diseases (e.g., foam cells with macrophages, etc.). That being said, the main catalyst of these diseases is inflammation, which then acts upon the specific cell type-giving rise to the clinical phenotypes. Serum PCSK9 concentrations have been reported to be positively correlated with inflammation, but PCSK9 has not been considered as a drug target with respect to inflammation to date.

Though these are primarily vascular diseases, PCSK9 is secreted mainly by the liver. There is limited secretion by other cell types, but the effects of extra-hepatic PCSK9 expression have not been demonstrated. Therefore, the hepatocyte targeted PCSK9 siRNA LNPs compositions and methods of the presently disclosed subject matter provide a new approach to managing inflammation and the myriad diseases, disorder, and conditions associated with inflammation.

However, downstream propagation of inflammation and other deregulated mechanisms within the vasculature remains therapeutically challenging. Accordingly, treatment and/or prevention of these diseases can also be accomplished with combination therapies that pair the LNPs of the presently disclosed subject matter with another drugs that targeting a converging mechanism in the cells specifically related to the particular disease, disorder, or condition of interest. Exemplary such approaches would be the use of a second active agent such as an antibiotic for the treatment of sepsis and/or for inhibiting the progression of sepsis to septic shock and/or cytokine storm. Similarly, a targeted LNP can be employed in treating and/or preventing atherosclerosis by, for example, inhibiting local vascular inflammation in macrophages, vascular endothelia, and smooth muscle cells via P-selectin targeting NLRP3 inhibitor nanoparticles. Although these two examples are merely exemplary of the use of the compositions and methods of the presently disclosed subject matter as part of a combination therapy, one of ordinary skill in the art would be able to select correct treatment modalities including particular drugs to pair the with LNPs of the presently disclosed subject matter as well as appropriate dosage ratios that are effective.

By way of example and not limitation, wherein the disease, disorder, or condition is an inflammatory disease, disorder, or condition, a LNP of the presently disclosed subject matter can be paired with a second active agent that is an anti-inflammatory agent, optionally a nonsteroidal anti-inflammatory drug (NSAID), a steroid, acetaminophen or other COX-3 inhibitor, a 5-lipoxygenase inhibitor, a leukotriene receptor antagonist, a leukotriene A4 hydrolase inhibitor, an antihistaminic, a histamine 2 receptor antagonist, a phosphodiesterase-4 antagonist, a cytokine antagonist, a CD44 antagonist, an antineoplastic agent, a 3-hydroxy-3-methyldutaryl coenzyme A inhibitor (e.g., a statin), an estrogen, an androgen, an antiplatelet agent, an antidepressant, a Helicobacter pylori inhibitor, a proton pump inhibitor, a thiazolidinedione, or a dual-action compound.

Also by way of example and not limitation, wherein the disease, disorder, or condition is a cardiovascular disease, disorder, or condition, optionally atherosclerosis, the LNPs of the presently disclosed subject matter can be paired with active agent that is an acyl-coenzyme A: cholesterol acyltransferase (ACAT) inhibitor, including but not limited to a selective inhibitor of ACAT-1, ACAT-2 as well as dual inhibitors of ACAT-1 and ACAT-2; an alpha-adrenergic blocking drug (alpha-blocker), an alpha/beta blocker, an angiotensin-converting enzyme (ACE) inhibitor, an aldosterone antagonist, an angiotensin 11 receptor antagonist, an anti-arrhythmic, an anticoagulant, an antiplatelet agent, an apolipoprotein A-1 (apoA-1) mimetic, a beta-blocker, a bile acid sequestrant, a calcium-channel blocker, an ApoB cholesteryl ester transfer protein (CETP) inhibitor, a cholesterol absorption inhibitor, a diuretic, a dyslipidemia agent, an endothelin receptor antagonist, a fibrate, a 3-hydroxy-3-methyl-glutaryl-coenzyme A (HMG-CoA) reductase inhibitor, an LCAT activator, an LDL receptor inducer, a lipase inhibitor, a lipoprotein-associated phospholipase A2 (Lp-PLA2) inhibitor, a microsomal triglyceride transfer protein (MTP) inhibitor; a platelet aggregation inhibitor, a PPAR agonist and/or activator, optionally a PPARγ agonist, PPARα agonist, and/or a PPAR dual α/γ agonists, a second PCSK9 antisense nucleic acid or RNAi, a squalene epoxidase inhibitor, a squalene synthetase inhibitor, a thrombolytic, a thyroid receptor beta activator, or any combination thereof. See e.g., U.S. Pat. No. 11,033,523, which is incorporated herein by reference in its entirety. In some embodiments, the disease, disorder, or condition is a cardiovascular disease, disorder, or condition, and the second active agent targets a vasculature cell, a muscle cell, a fibroblast, and/or an endothelial cell, optionally a vascular endothelial cell, wherein the second active agent binds to a target selected from the group consisting of LOX-1, a selectin, optionally a P-selectin or an E-selectin, an integrins, and a neuropilin.

Also by way of example and not limitation, wherein the disease, disorder, or condition is sepsis, septic shock, or cytokine storm, the second active agent paired with an LNP of the presently disclosed subject matter can be an antibiotic, a complement inhibitor, optionally a complement inhibitor selected from the group consisting of a C5a inhibitor, a C5aR inhibitor, a C3 inhibitor, optionally wherein the C3 inhibitor is Compstatin, a Compstatin analog, a Compstatin peptidomimetic, or a Compstatin derivative, a factor D inhibitor, a factor B inhibitor, a C4 inhibitor, a C1q inhibitor, or any combination thereof. See e.g., U.S. Pat. No. 7,968,522, which is incorporated herein by reference in its entirety.

It is noted that the biological pathways disclosed with particularity herein are meant to be exemplary only, and other biological pathways that are associated with diseases, disorders, and conditions can be modulated with the compositions and methods of the presently disclosed subject matter as part of a combination therapy. Exemplary such pathways are those associated with undesirable levels of expression of CD36, TLR-4, TN-F-α, IL-6, NLRP3.

Accordingly, in some embodiments the presently disclosed subject matter relates to methods for delivering active agents comprising LNPs to cells, tissues, and organs including but not limited to hepatocytes in order to inhibit biological activities that result from undesirable gene expression in the cells, tissues, and organs to thereby treat and/or prevent diseases, disorders, and/or conditions associated with the undesirable levels of PCSK9 gene expression in cells, tissues, and/or organs. In some embodiments, the LNPs of the presently disclosed subject matter are contacted with cells in vitro or ex vivo and in some embodiments the LNPs of the presently disclosed subject matter are administered to subjects in amounts and via a route that is effective to treat and/or prevent diseases, disorders, and/or conditions associated with the undesirable levels of PCSK9 gene expression in cells, tissues, and/or organs of the subject including but not limited to hepatocytes.

### EXAMPLES

The following EXAMPLES provide illustrative embodiments. In light of the present disclosure and the general level of skill in the art, those of skill will appreciate that the following EXAMPLES are intended to be exemplary only and that numerous changes, modifications, and alterations can be employed without departing from the scope of the presently disclosed subject matter.

Without further description, it is believed that one of ordinary skill in the art can, using the preceding description and the following illustrative EXAMPLES, make and utilize the compounds of the presently disclosed subject matter and practice the methods of the presently disclosed subject matter. The following EXAMPLES therefore particularly point out embodiments of the presently disclosed subject matter arid are not to be construed as limiting in any way the remainder of the disclosure.

### Materials and Methods for the EXAMPLES

Materials. All ionizable lipids, including but not limited to DLin-MC3-DMA ((6Z,9Z,28Z,31Z)-Heptatriaconta-6,9,28,31-tetraen-19-yl 4-(dimethylamino)butanoate; also called MC3), were purchased from Cayman Chemical (Ann Arbor, Michigan, United States of America). All other lipids were purchased from Avanti Polar Lipids, Inc. (Alabaster, Alabama, United States of America). PCSK9 siRNA (sense: GGCAUUCAAUCCUCAGGUCtt (SEQ ID NO: 7), antisense: GACCUGAGGAUUGAAUGCCtg; SEQ ID NO: 8) was obtained from BOC Sciences (Shirley, New York, United States of America). The LNPs were fabricated using the NANOASSEMBLR® Benchtop from Precision Nanosystems (Vancouver, British Columbia, Canada). Dialysis cassettes were obtained from Thermo Fisher Scientific (Waltham, Massachusetts, United States of America). Ethanol and PBS were obtained from Fisher Scientific (Waltham, Massachusetts, United States of America) and citrate was obtained from Sigma-Aldrich, Inc. (St. Louis, Missouri, United States of America). SLS and zeta potential was determined using a Malvern Zetasizer (Malvern Panalytical Inc., Westborough, Massachusetts, United States of America). For flow cytometry, the ATTUNE™ Nxt (Thermo Fisher Scientific) was used.

LNP Formulation. Stock solutions of the lipids were prepared beforehand and were diluted with 100% ethanol based on the lipid stock solutions. Other solvents including but not limited to Butanol, IPA, methanol, acetone, acetonitrile can be used to dissolve lipids.

LNP Lipid Formulation. After the stock solutions had been prepared, the lipid mixes were made based on the mole fractions of lipids, shown in the Tables below, Then lipid mixes were diluted with 100% ethanol to final total lipid constituent concentration of 3 mM, Other solvents including but not limited to Butanol, IPA, methanol, acetone, acetonitrile can be used to further dilute the lipid mixes.

siRNA Dilution. The siRNAs were rehydrated with RNAse free water to achieve a concentration of 1 mg/mL. Nucleic acid quantification was then performed via the TAKE3™ plate RNA quantification function on the BioTek CYTATION™ 3 plate reader (Agilent Technologies, Inc., Santa Clara, California, United States of America). The readouts of siRNA concentration were used to calculate the volume of citrate buffer needed to dilute the samples to the desired siRNA concentration of 0.25 mM.

### Example 1

### Microfluidic Assembly

Precision Nanosystem's NANOASSEMBLR® and microfluidic chips were used in order to create the LNPs. The parameters that were used can be seen below: Flow Rate Ratio=3 aqueous: 1 organic, Start Waste=0.2.5 mL, End Waste=0.0.5 mL, Total Flow Rate=12 mL/min, First, one of the ethanol compatible chips was first placed into the NANOASSEMBLR®. Next, a priming run was done using 1.5 mL of pH 4.5 citrate buffer in a 3 mL syringe, which was inserted into the left/aqueous syringe hole, and 0.5 mL of 100% ethanol in a 1 mL syringe, which was inserted into the right/organic syringe hole. The total volume on the NANOASSEMBLR® system was set to 2 mL and allowed to run. For LNP assembly, 375 μL of the siRNA citrate buffer solution was taken up in a 3 mL syringe and placed in the left aqueous syringe hole. Additionally, 125 μL of the lipid mix was taken up in a 1 mL syringe and placed in the right/organic syringe hole. The total volume was set to 0.5 mL and the NANOASSEMBLR® was allowed to run. All formulations have a ghost (no siRNA) as a control, containing the same lipid mole fractions as their siRNA counterparts.

### Example 2

### LNP Dialysis

Both generic and Inclisiran LNP samples were then put through dialysis using 0.5 mL dialysis cassettes placed in 250 mL 1× PBS. After 2 hours, the samples were removed and characterization and. siRNA quantification were performed.

### Example 3

### Dynamic Light Scattering (DLS) and Nanoparticle Tracking Analysis (NTA)

A Malvern Zetasizer (Malvern Panalytical Inc.) was used to determine the size and zeta-potential of the formulations. In a plastic cuvette, 20-50 μL LNPs and a water or PBS were mixed to get a total volume of 500 μL. The standard particle size SOP was used. For the zeta potential, 20-50 μL of LNPs and the volume of water or PBS to achieve a total volume of 600 μL were added to a folder capillary zeta cell cuvette. The standard zeta-potential SOP was performed.

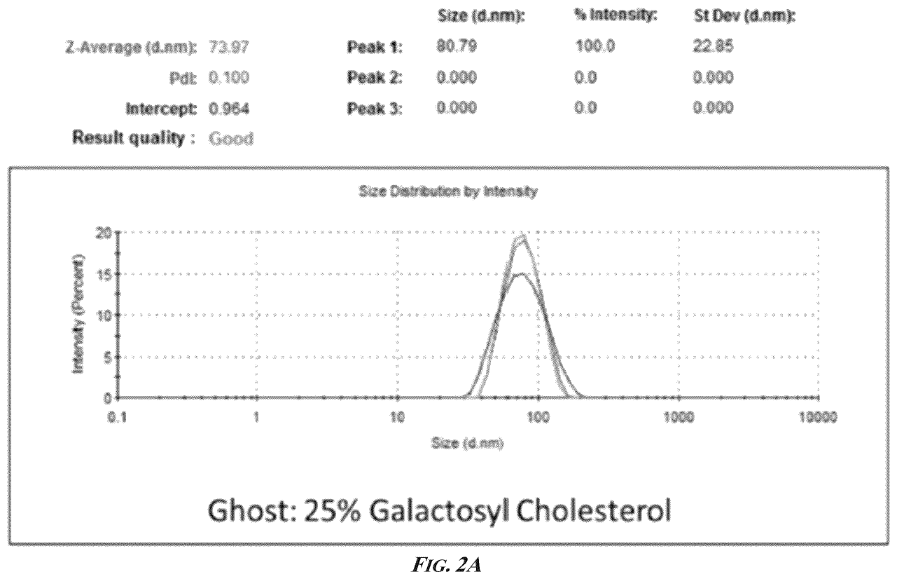

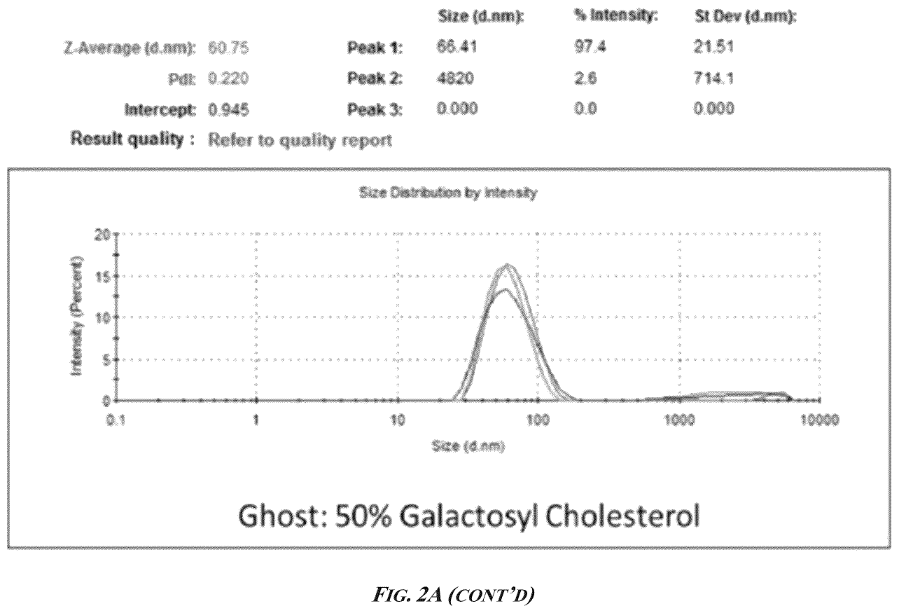

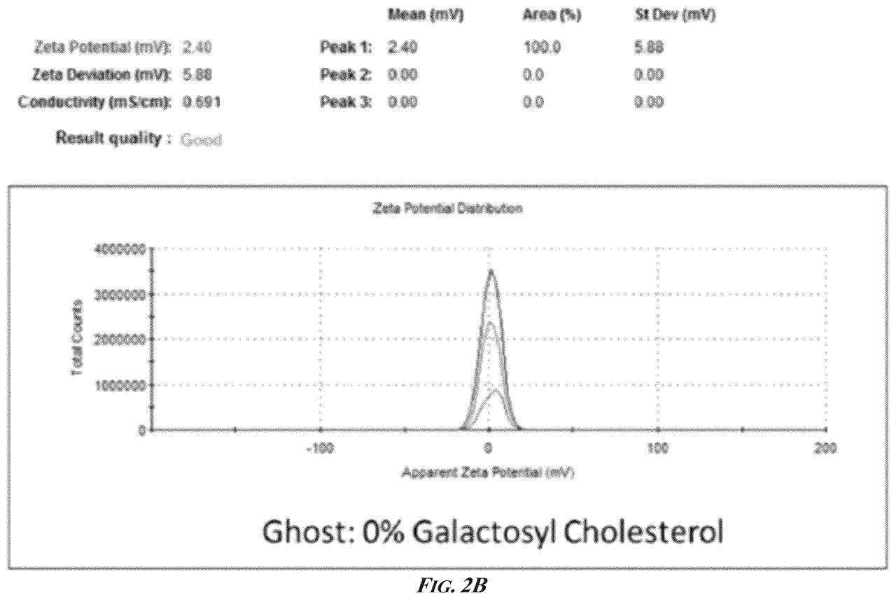

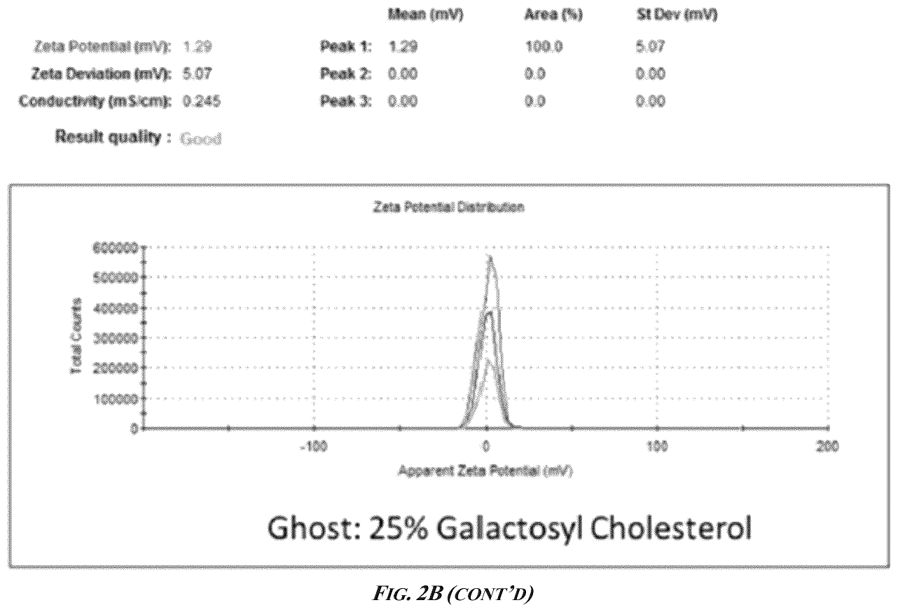

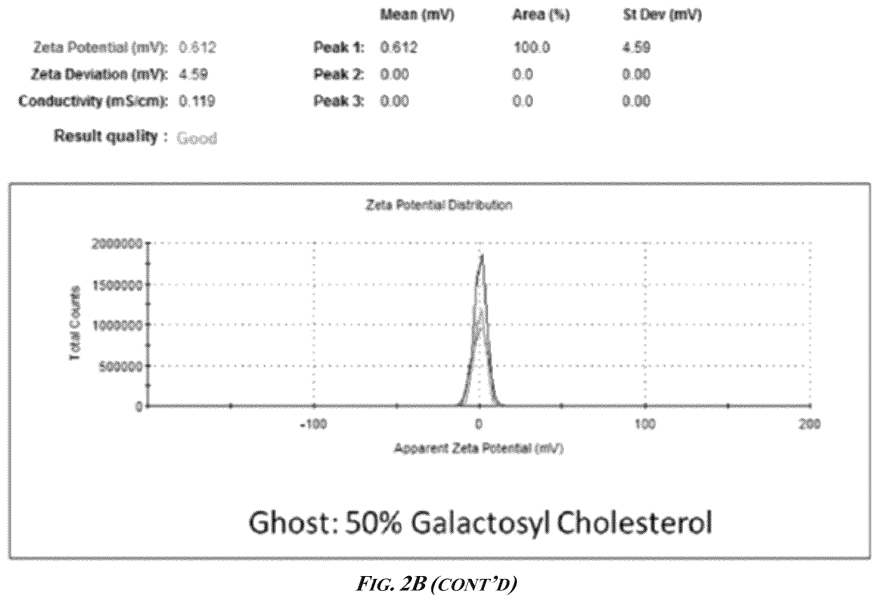

Exemplary DLS analyses are shown in FIGS. 1A-3C.

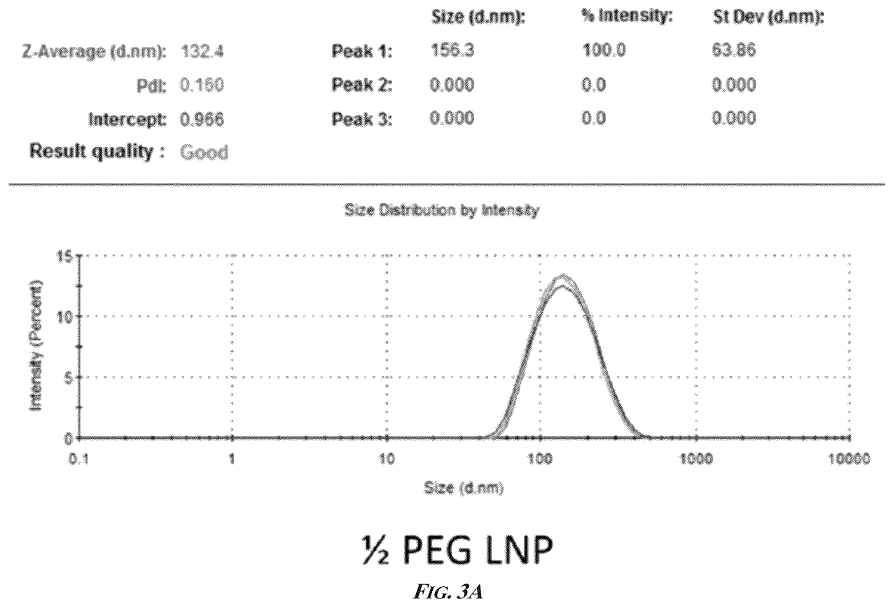

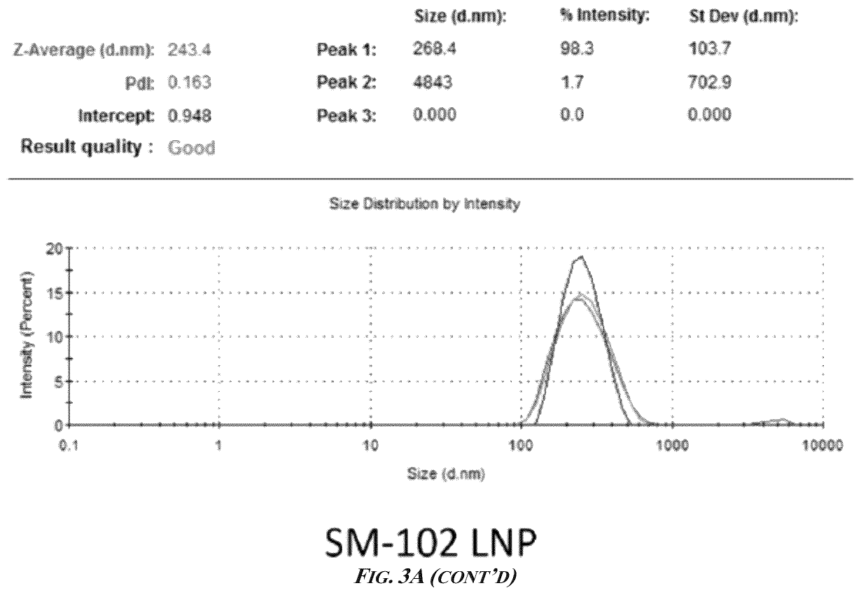

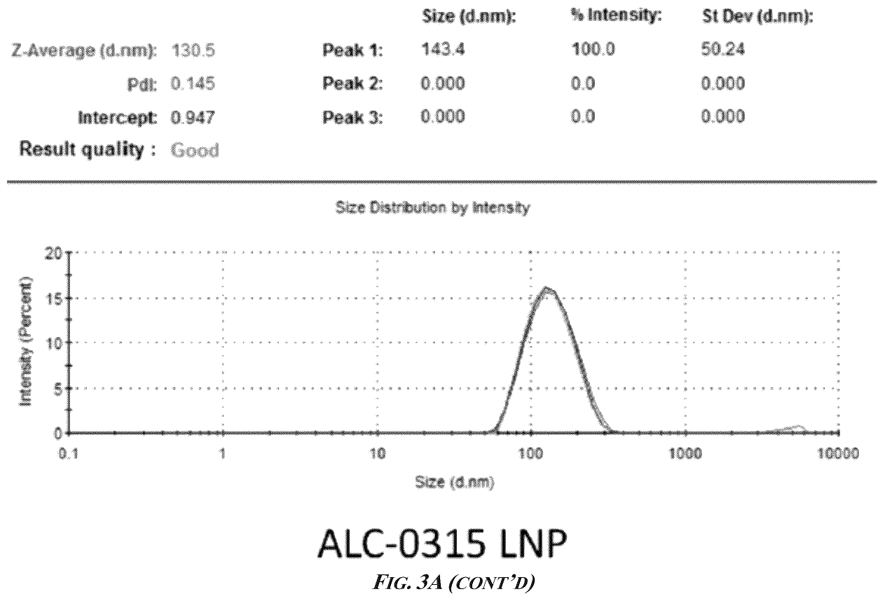

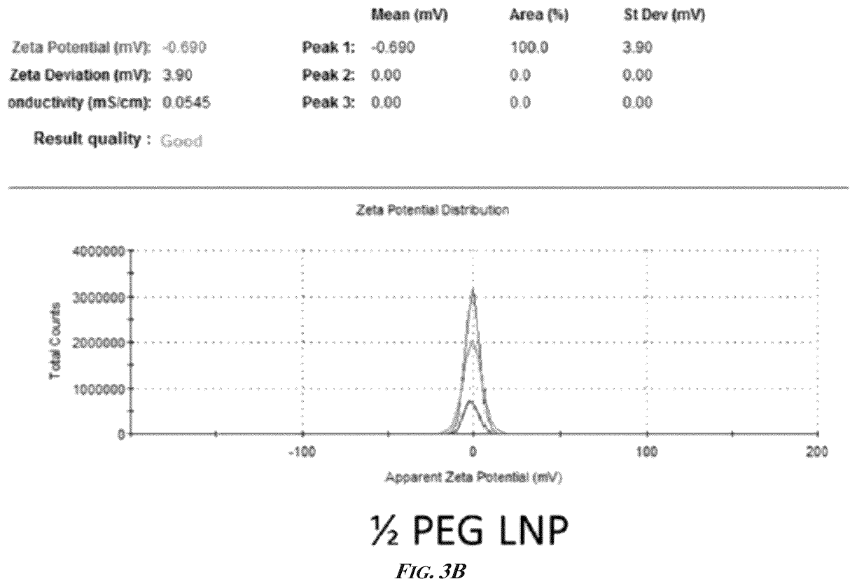

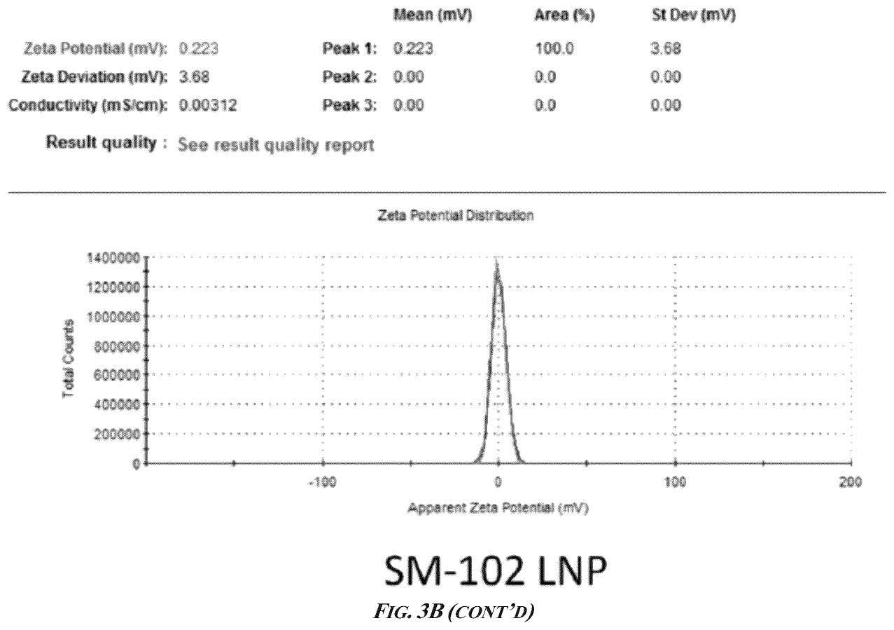

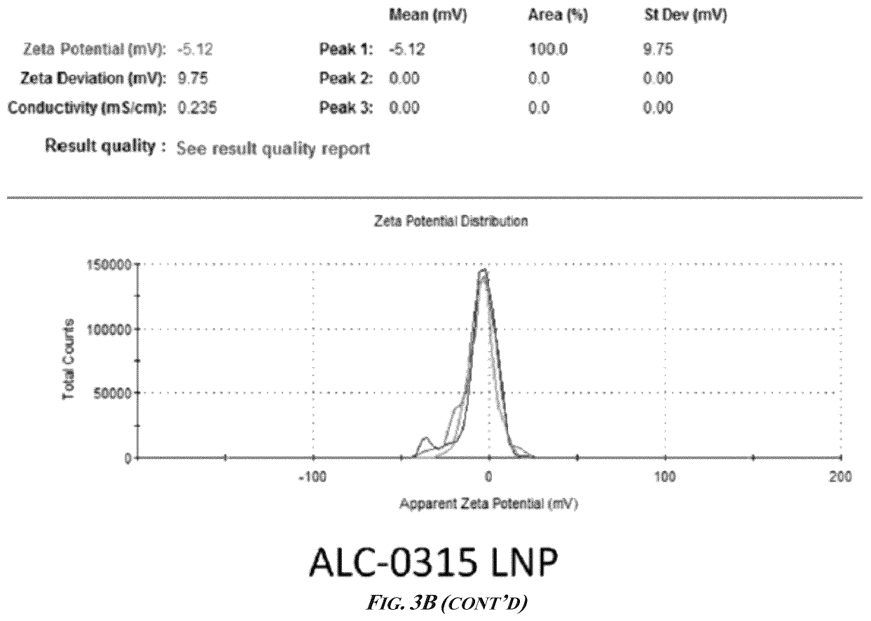

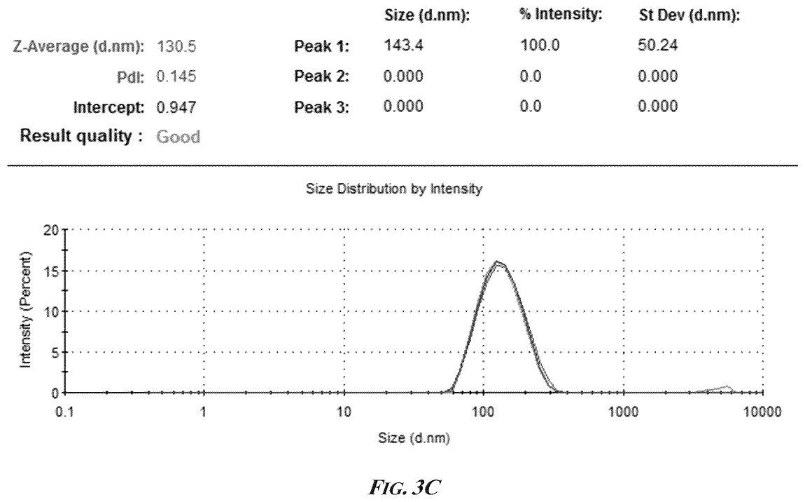

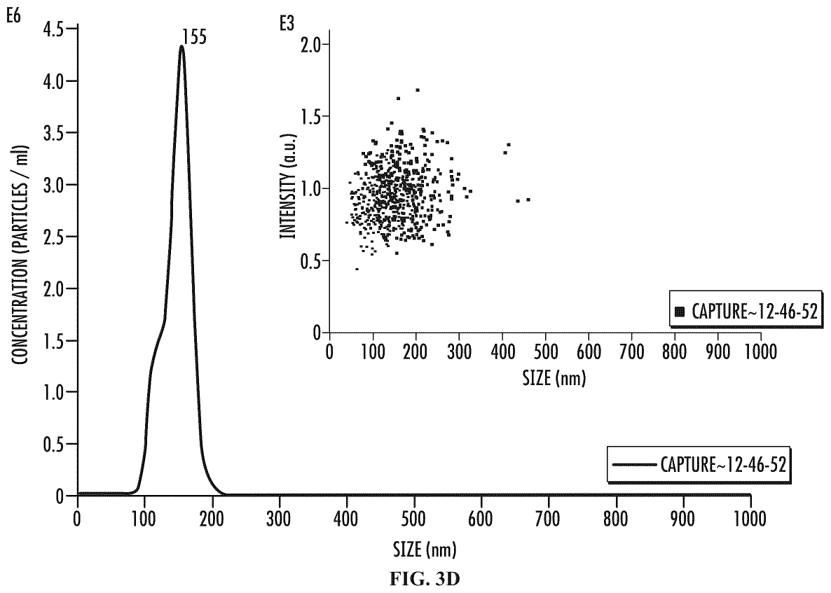

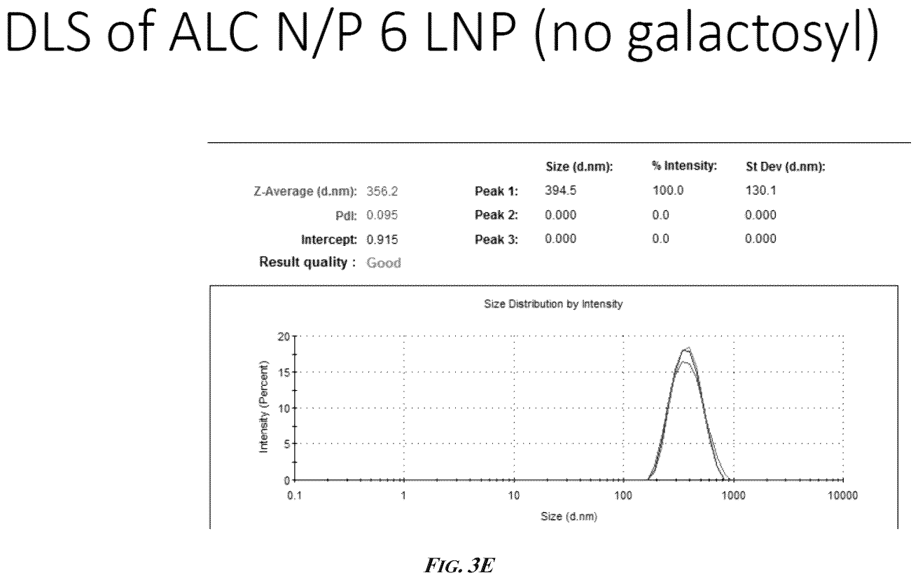

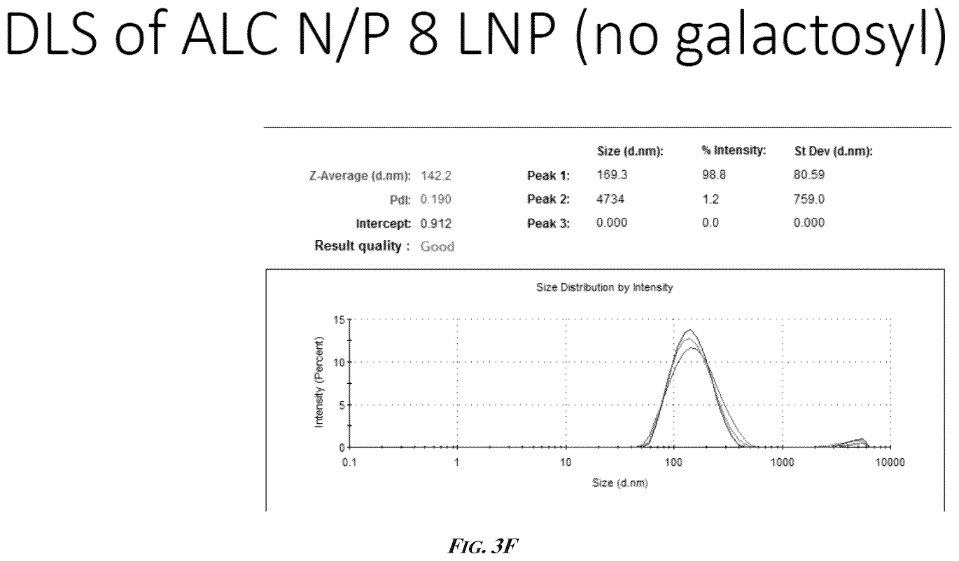

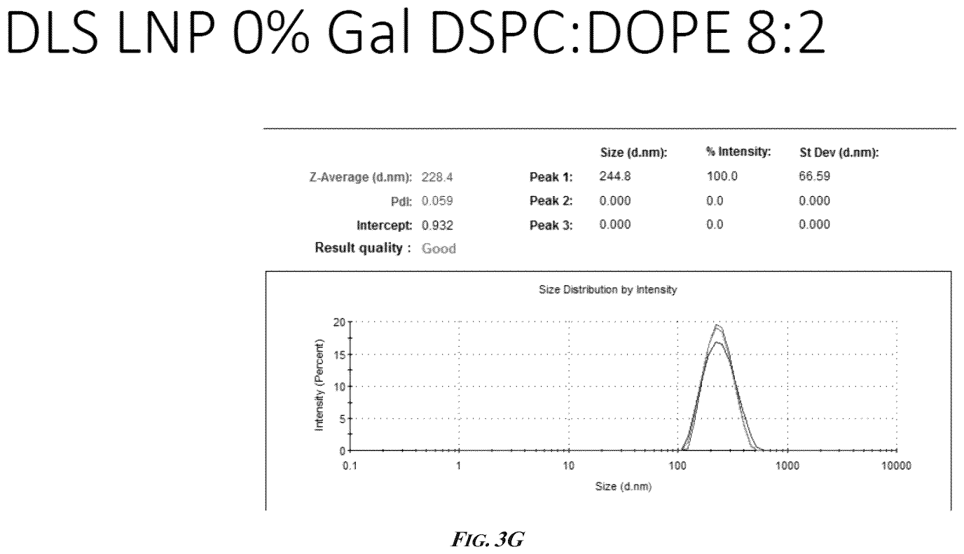

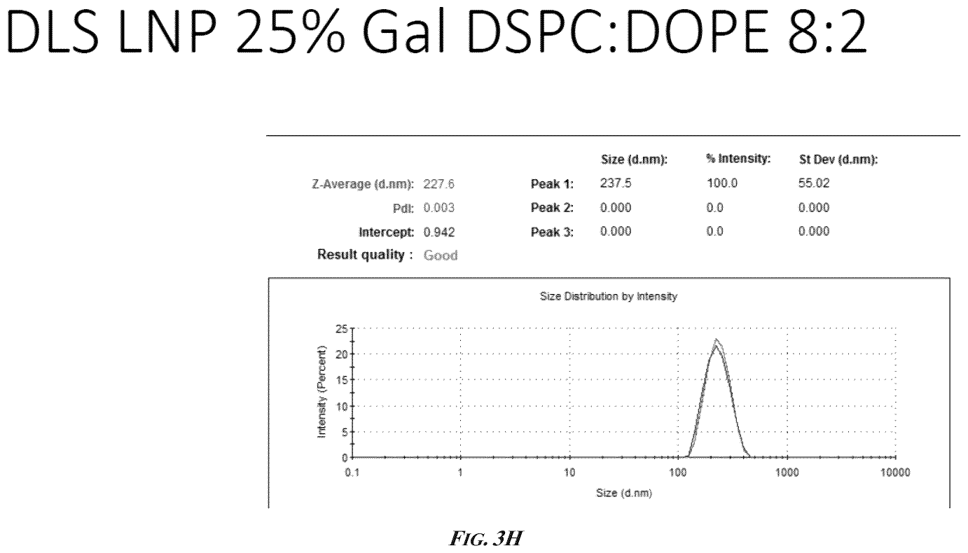

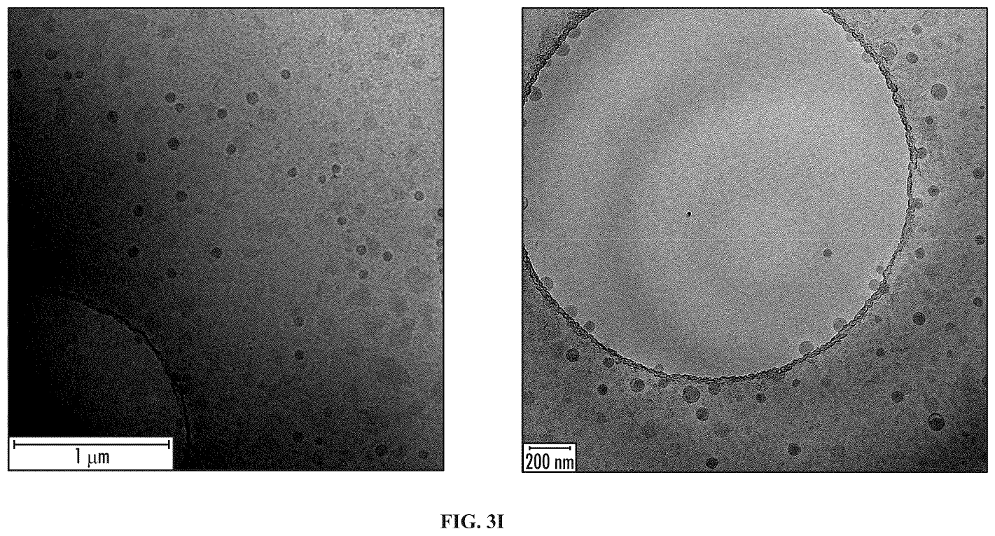

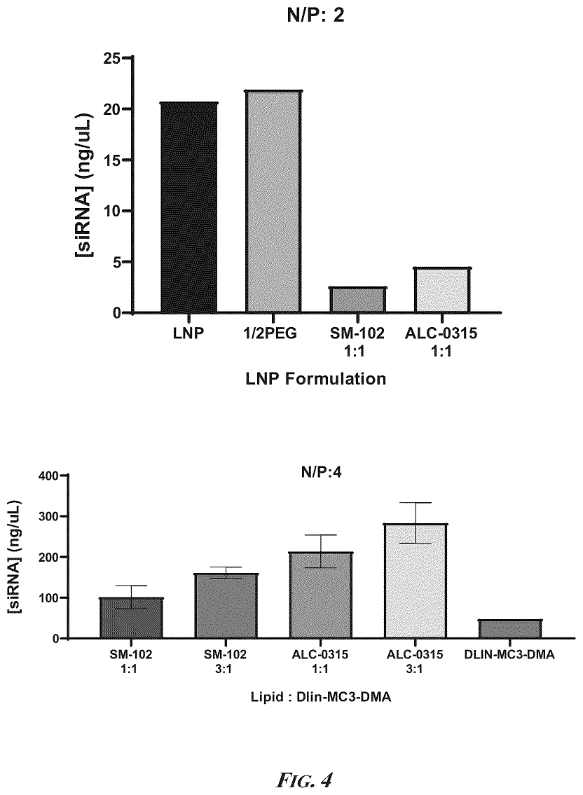

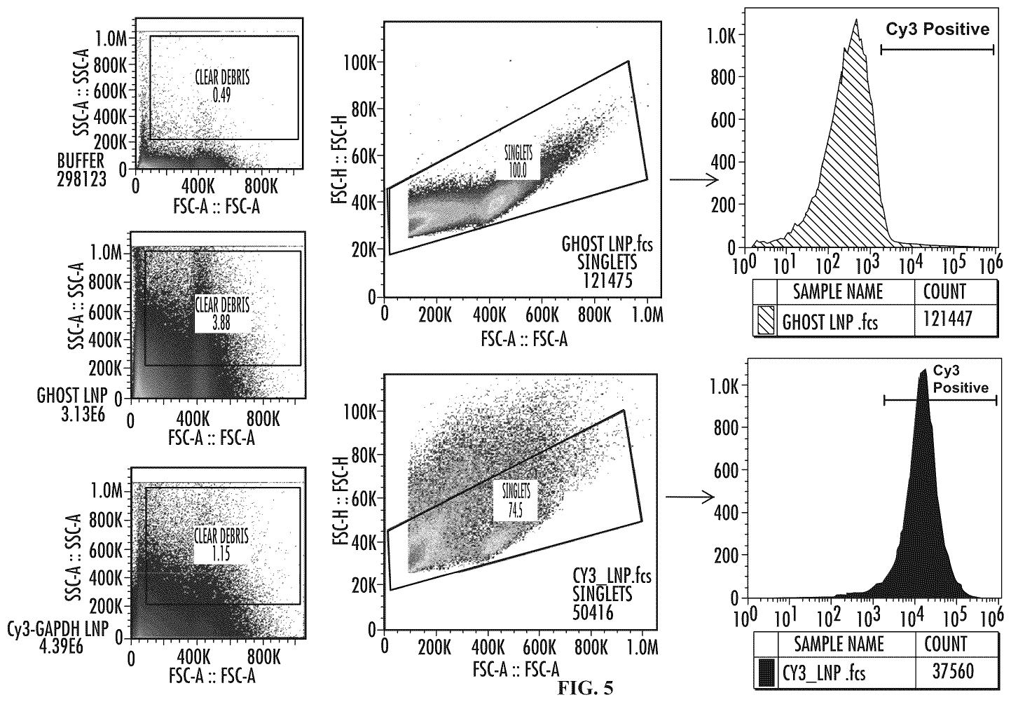

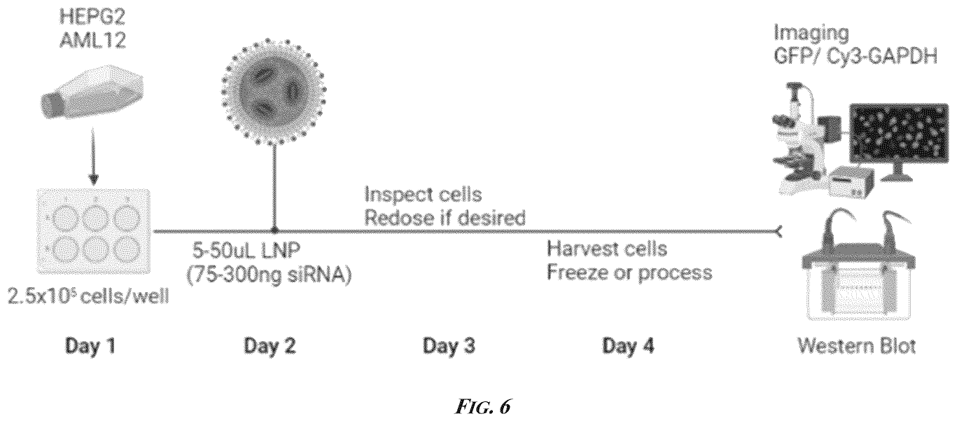

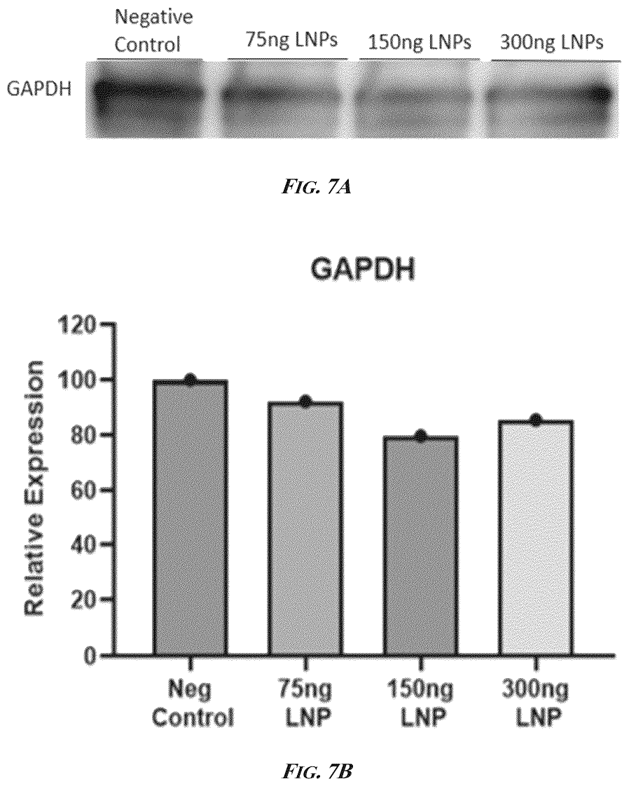

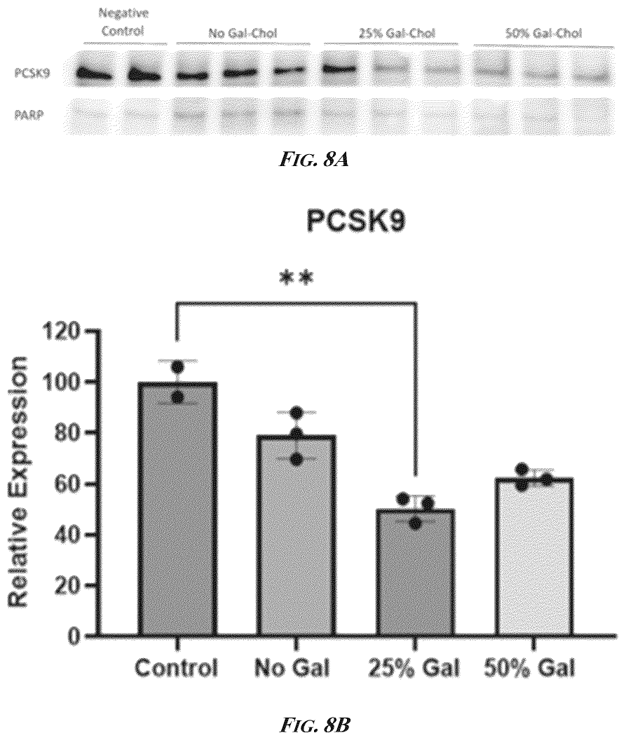

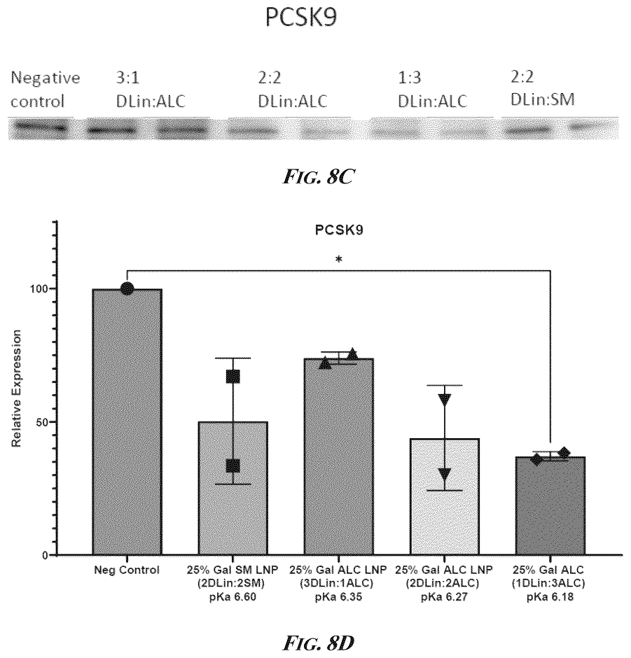

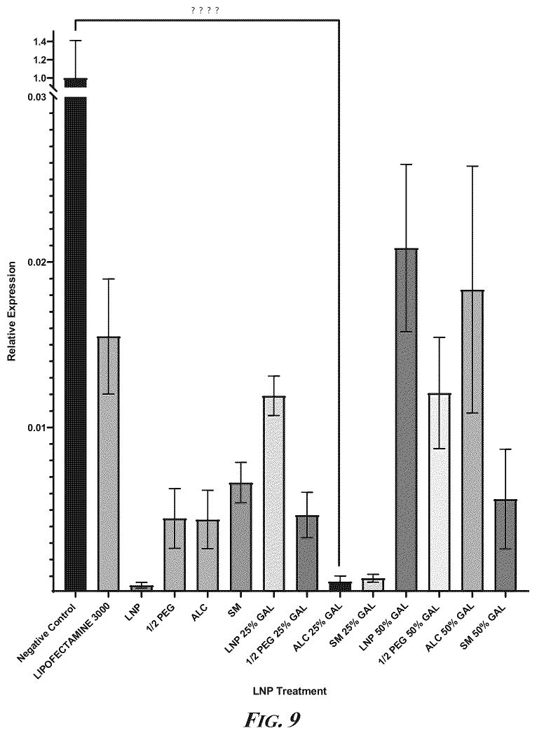

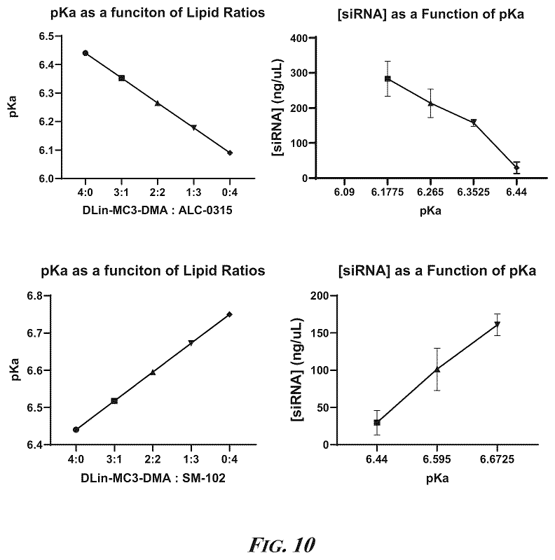

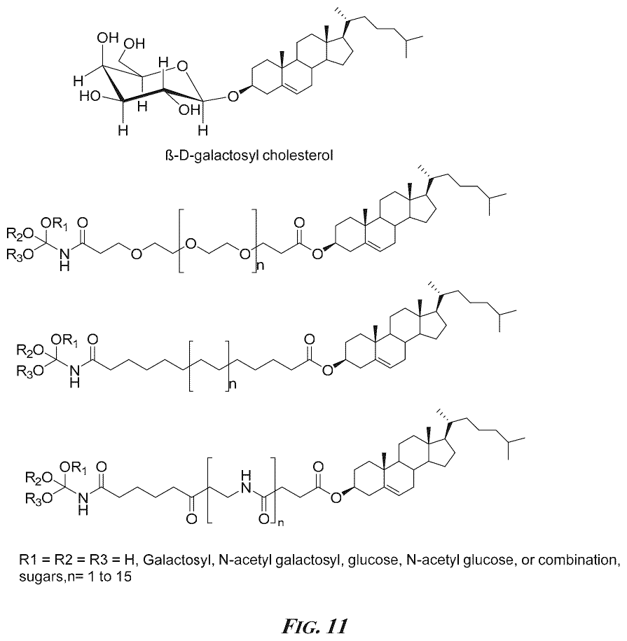

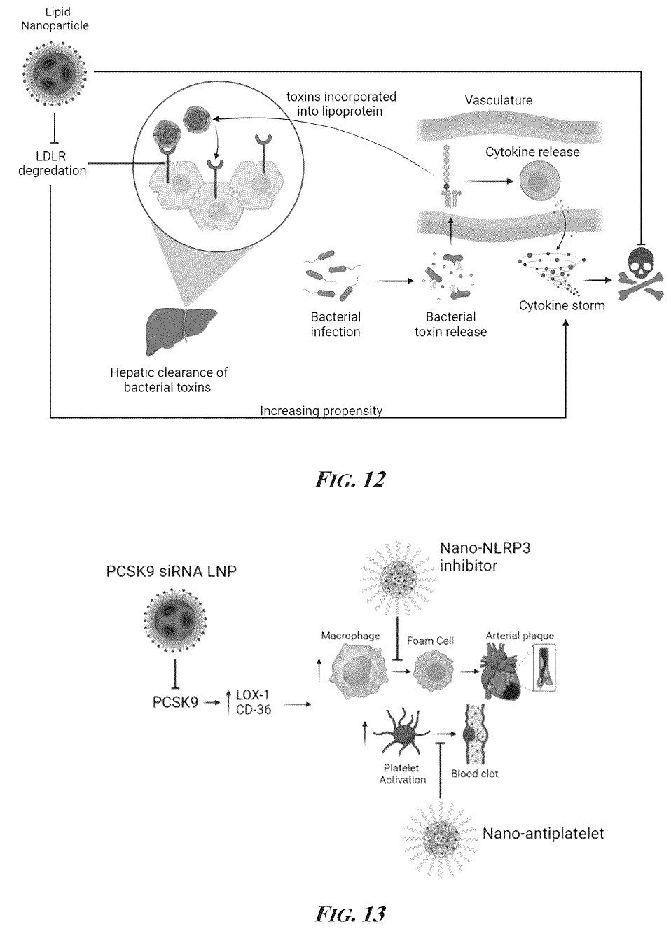

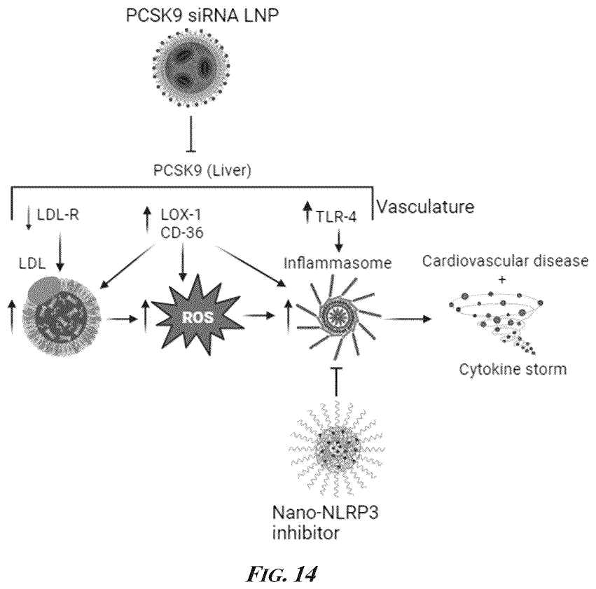

FIG. 3D provides Nanoparticle Tracking Analysis (NTA) data of ALC 1:1 Dlin 25% galactosyl-cholesterol LNP.

FIG. 3E provides cryo-electron microscopy analysis of LNPs to visualize NP morphology and size distributions. These images show a relatively monodisperse LNP both in ghost and siRNA loaded NPs.

### Example 4

### siRNA Quantification

The RIBOGREEN® assay (Promega Corporation, Madison, Wisconsin, United States of America) was used. In row A of a 96 well plate 235 μL 1× TE buffer and 15 μL LNP sample or PBS blank was added. 50 μL of 1× TE buffer was added to rows B/C (replicates) with 50 μL of the corresponding row A sample TE buffer mixture (ex. 50 μL A1 distributed to B1 and C1 respectively A2 to B2 and C2 and so on). In rows C/D, 50 μL 1× TE with 1% 100× Triton was added and 50 μL of the subsequent row A mixture. Rows F/G calibration curve, an RNA standard was prepared by adding 100, 50, 10, 2, 0 μL 100 ng/mL RNA stock with 0, 50, 90, 98, 100 μL 1× TE respectively. The well plate was incubated at 37° C. for 10 minutes. To each well in rows F/G, 50 μL Triton+TE was added. The RIBOGREEN® reagent was diluted 2000-fold in TE buffer and 100 μL was added to each well the plate was incubated at RT for 5 minutes. Then a needle was used to pop any bubbles formed. The fluorescence was read with a plate reader with an excitation wavelength of 485 nm, emission wavelength at 528 nm, read height of 8 mm and gain of 55. Before making the calibration curve, first the replicate rows were averaged, then background from PBS samples subtracted from the fluorescence values.

### Example 5

### Cell Culture and Western Blot

293T, HepG2, and AML12 cells were cultured in DMEM+10% FCS to no more than 80% confluency. Other cell lines can be used including but not limited to HepG2, Hep3B, HepT1, HuH6, HuH7, AML12 (mouse), PCL/PRF/5, PHH, 1-7-1, SMMC-7721. These additional cell lines accommodate other disease models including but not limited to HCC grades I-III, NAFLD/NASH, AFLD, Hepatic cirrhosis, Chronic active hepatitis, Viral Hepatitis A-E, and Epstein Barr virus. For a 6 well plate, each well was plated to ˜250,000 cells per well in 2-3 mL of media. Dosing the LNPs was done between 600-75 ng siRNA/mL of media. 48-72 hours after the previous dose, cells were dislodged, collected, and centrifuged to pellet at 500 ref for 3 minutes, Then media was aspirated and cells were washed with 1× PBS and again pelleted. To the cells, 250 μL RIPA buffer was added and then cells were sonicated at 35% amplitude in three 4 second cycles. After, cells were centrifuged at 12,000 ref at 4° C. for 10 minutes. After which, the DC protein assay was performed to determine the concentration of the sample to add to the 12% mini-PROTEAN TGX STAIN-FREE™ Gels (Bio-Rad Laboratories, Hercules, California, United States of America). Samples were loaded on SDS-PAGE gel and samples resolved at 200V for 30 minutes. After, proteins were transferred to a PVDF membrane via the TRANS-BLOT® TURBO™ Transfer System (Bio-Rad). Then, the membrane was blocked by rocking for an hour at room temperature with casein or 5% BSA in TBS-T. Then, the blocking buffer was discarded and the primary antibody, diluted 1:1000 in 5% BSA in TBS-T, was added to the membrane and incubated with rocking overnight at 4° C. After incubation, the membrane was washed 3 times by rocking the membrane for 5 minutes with TBS-T. Then, the corresponding HRP conjugated secondary antibody, diluted 1:10,000, was added to the membrane and was allowed to rock for 1 hour at RT. Then, membrane washing was again completed, and after which, the membrane was added to a black reflective screen, and Excellent Chemiluminescent Substrate (ECL) reagents (ELABSCIENCE® Biotechnology Inc., Houston, Texas, United States of America) were mixed and pipetted onto the membrane. Then the screen was placed into a GeneSys Imaging system (Syngene, Frederick, Maryland, United States of America) and was imaged with no light for 6.5 minutes.

### Example 6

### Flow Cytometry

Cy3-GAPDH siRNA encapsulated LNPs were diluted fourfold with PBS to 200 μL final volume. Then, 400 μL of flow cytometry staining buffer was added to this LNP solution. Samples were collected on Attune NXT flow cytometry (Life Technologies). LNPs were gated on FSC-A/SSC-A to exclude debris in the sample and Cy3 fluorescence was examined by gating on LNP singlets, as described in the FIG. 5 Positive Cy3 fluorescence indicates successful encapsulation of Cy3-GAPDH siRNA in LNPs. The flow cytometry data was analyzed using FlowJo software (version 10.5.3) (Ashland, OR).

### Example 7

### RT-qPCR

Cells were treated with siRNA containing LNPs and/or controls for 48 h then collected by aspirating the media and washing lx with PBS. Plates of cells were then frozen or immediately processed to isolate RNA using Trizol based extraction. In brief, 1 mL of Trizol was added per well and lysis was allowed to continue for 5 minutes. Lysate was transferred to a 1.7 ml tube, combined with 200 ul of chloroform and vortexed for 2 minutes. Tubes were then centrifuged at 12,000×g for 15 minutes at 4C, resulting in the formation of three lavers. The top layer was transferred to a fresh 1.7 ml tube and combined with 500 ul of isopropanol. These new tubes were centrifuged at 12,000×g for 10 minutes at 4C. The isopropanol was discarded and RNA pellets were resuspended in 1 ml of −20C 75% EtOH and centrifuged again at 7500 x g for 5 minutes at 4C. The EtOH was removed and the final RNA pellet was resuspended in H2O and quantified using a NanoDrop Instrument. 2000 ng of RNA was taken from each sample and processed to form cDNA using the Bio-Rad iScript cDNA synthesis kit. To perform qPCR, 2 ul of this cDNA was added to a Bio-Rad PCR plate along with 0.5 ul of target specific Taqman probe, 5 ul of Bio-Rad Probe Supermix and 2.5 ul of H2O. The plate was then sealed and imaged using the standard Bio-Rad Probe protocol (39 cycles, imaging after every cycle). The data was normalized to reference probes (PSMB6, B2M and/or HPRT1) followed by normalization to control.

### Example 8

### Production of an siRNA-Containing LNP

To create the LNP, the amphiphilic ionizable lipid D-Lin-MC3-DMA, the charge of which changes as a function of pH, was employed in place of traditional cationic lipids in order to provide decreased toxicity, decreased macrophage activation, increased endosomal escape, and neutral zeta potential when compared to traditional cationic lipids, DLin-MC3-DMA contains hydrolysable ester linkages that generally promote physiological degradation of the lipid and to create an efficacious siRNA delivery material. Other ionizable and cationic lipids were incorporated, such as SM-102 and ALC-0135 respectively, to further aid in endosomal escape. Efficient LNP drug delivery occurs when three LNP structural criteria are met: having a secondary or tertiary amine head group, a hydrophobic 12-18 length carbon chain linked by a hydrolysable ester group, and an ionizable lipid pKa of 6-6.8. We used a ratiometric approach to combine existing FDA approved hydrolysable lipids (pKa range, 6.0-6.75), DLinMc3DMA (pKa, 6.44), ALC-0315(pKa, 6.09) and SM-102 (pKa, 6.75) to achieve the optimal lipid dissociation constant, pKa of 6.0-6.44. Mixing ionizable lipids to manipulate pKa was critical to improving the drug potency, modulating tissue specific delivery, and transfection efficiency of LNPs. These nanoparticles organize into core shell structures. Core consists of electrostatically bound RNA with ionizable lipids, and cholesterol contributes integrity. DSPC and PEG reside in the outer layer and form the nanoparticle shell. The helper lipids employed included cholesterol, DSPC, and DMG-2000-PEG, which achieved ˜100-200 nM size particles. DMG-PEG allows for in vivo stealth attributed by PEG; DMG is a neutral lipid similar in structure to DSPC, which is an endogenous helper lipid. DOPE lipid contains an unsaturated chain and adding fluidity and stability to the particle. DOPE may improve the transfection. Cholesterol, naturally occurring P-sitosterol, and other naturally occurring or synthetic cholesterol analogs improve RNA delivery, cellular uptake, structural stability to the LNP, and serves as the exemplified conjugate for the galactosyl (Races et al., 2020; Zhang et al., 2022). Targeting sugars such as monomeric sugar moieties, N-acetylated sugar moieties, or multiple sugar moieties can be conjugated to the head/tail sections of cholesterol and PEG. The linker size may be 10-15 carbon chain lengths of hydrophobic fatty acids, hydrophilic glycols, and linked either through ester or amide bond to improve the binding efficiency of targeting ligand with the chosen, target receptors.

The presently disclosed LNPs have the added property of targeting through intercalation of galactosyl-cholesterol into the LNP. We developed a ‘platform technology’ to deliver various therapeutic siRNA to the targeted tissue. A fluorescent dye known as DiO was also incorporated. The presently disclosed LNPs had the added property of targeting through intercalation of galactosyl-cholesterol into the LNP.

LNPs were prepared by microfluidic mixing of ethanol dissolved lipids and 4.5 pH citrate buffered siRNA on a Benchtop Precision NANOASSEMBLR®. Purification of targeted LNP was achieved by using dialysis cassettes at 7.4 pH, Charged lipids surround the RNA backbone forming a complex inside the LNP which holds the LNP together and protects the siRNA; this role is performed by DLin-MC3-DMA, ALC0315, and SM-102 which are ionizable lipids that are cationic in the citrate buffer. This also benefits endosomal escape, as its pH decreases, ionizable lipids become cationic and facilitate siRNA cytosolic delivery through destabilization of the endosomal membrane via the proton sponge effect. DLin-MC3-DMA containing LNPs are being used in multiple clinical trials (Flu et al., 2020; Zhang et al., 2022).

As a proof of concept, a carbohydrate conjugated-LNP system was engineered as cargo for siRNA delivery to hepatocytes.

### Example 9

### LNP Treatment to Treat Sepsis and Cytokine Storm

FIG. 12 provides a schematic representation of how the LNPs of the presently disclosed subject matter can be used to treat sepsis and/or inhibit cytokine storm or its consequences. First, bacterial products enter the bloodstream, where they are incorporated into LDL particles thereby requiring LDLR mediated heroic: clearance. If PCSK9 is present, this clearance downregulates the LDLR making it harder to clear pro-inflammatory bacterial products, leading to or exacerbating cytokine storm. When the LNP particles target hepatic PCSK9 translation, the LDLRs can be recycled, reducing the buildup of cytokine storm inducing elements.

### Discussion of the EXAMPLES

Disclosed herein is a “platform technology” to deliver therapeutic siRNAs to targeted cells, tissues, and organs. A carbohydrate conjugated-LNP system was engineered as cargo for siRNA delivery to hepatocytes by utilizing ionizable cationic lipids, phospholipids, sugar-conjugated cholesterol, and PEG-lipids to generate ˜100 nM sized particles with narrow size distributions (FIGS. 1-3), LNPs were prepared by microfluidic mixing of ethanol-dissolved lipids and pH 4.5 citrate-buffered siRNA on a Benchtop Precision NANOASSEMBLR®. Purification of targeted LNP was achieved using y dialysis cassettes at 7.4 pH. The physiochemical properties were measured on a Malvern Light scattering instrument. A modified siRNA sequence to silence PCSK9 was obtained from BOC Sciences as a HPLC purified, lyophilized powder, and was stored at −20° C. The modified siRNA sequence molecular weight 14550.7 daltons (Da)) was composed of the following:

| | |
|---|---|
| sense strand | cs-us-a-g-a-c-Cf-u-Gf-u-dT-u-u-g-c-u-u-u-u-g-u, |
| | molecular weight 6852.51 Da (SEQ ID NO: 1) |
| antisense strand | as-Cfs-a-Af-Af-Af-g-Cf-a-Af-a-Af-c-Af-g-Gf-u-Cf-u-a-gs-as-a; molecular |
| | weight 7698.17 Da (SEQ ID NO: 2) |

a: 2′-O-methyladenosine-3′-phosphate; c: 2′-O-methylcytidine-3′-phosphate; g: 2′-O-methylguariosine-3′-phosphate; u: 2′-O-methyluridine-3′-phosphate; cs: cytidine-3′-phosphorothioate; us: 2′-O-methyluridine-3′-phosphorothioate, Cf: 2′-fluorocytidine-3′-phosphate; Gf: 2′-fluoroguanosine-3′-phosphate; dT: 2′-deoxythymidine; as: 2′-O-methyladenosine-3′-phosphorothioate; Cfs: 2′-fluorocytidine-3′-phosphorothioate; Af: 2′-fluoroadenosine-3′-phosphate; gs: 2′-O-methylguanosine-3′-phosphorothioate.

LNPs were used to treat cells in vitro to knockdown target mRNAs in a dose dependent and targeting ligand dependent manner, measured by qRT-PCR and western blot (FIGS. 7-9). Transfection was highly formulation, size, and surface charge dependent (see FIGS. 1-3). Flow cytometry was used to ensure an equal siRNA distribution per particle as well as determine if there area sufficient number of particles per cell according to our experimental design (FIG. 5). The RIBOGREEN® assay was used to quantify the encapsulated siRNA in order to correctly dose the cells (FIG. 4).

TABLE 1
LNP Formulation Without the Targeting Moiety

| Lipid | MW (mg/mL) | Molar Ratio |
|---|---|---|
| DSPC | 79.0161 | 0.1 |
| DLin-MC3-DMA | 642.1 | 0.5 |
| DMG-PEG(2000) | 2509.2 | 0.015 |
| Cholesterol | 386.64 | 0.375 |
| DiO* | 825.6 | 0.01 |

*in some embodiments, DiO is omitted

TABLE 2
LNP Formulation with 25% Gal-Chol Calculated
from the Cholesterol Mole Fraction

| Lipid | MW (mg/mL) | Molar Ratio |
|---|---|---|
| DSPC | 790.16 | 0.1 |
| DLin-MC3-DMA | 642.1 | 0.5 |
| DMG-PEG(2000) | 2509.2 | 0.02 |
| Cholesterol | 386.64 | 0.28125 |
| Galacatosyl Cholesterol | 548.79 | 0.09375 |
| DiO* | 825.6 | 0.01 |

*in some embodiments, DiO is omitted

TABLE 3
LNP Formulation with 50% Gal-Chol Calculated
from the Cholesterol Mole Fraction

| Lipid | MW (mg/mL) | Molar Ratio |
|---|---|---|
| DSPC | 790.16 | 0.1 |
| DLin-MC3-DMA | 642.1 | 0.5 |
| DMG-PEG(2000) | 2509.2 | 0.02 |
| Cholesterol | 386.64 | 0.14 |
| Galactosyl Cholesterol | 548.79 | 0.19 |
| DiO* | 825.6 | 0.01 |

*in some embodiments, DiO is omitted

TABLE 4
LNP Formulation with Half Mole Fraction of PEG

| 1/2 PEG Lipid | MW (mg/mL) | Molar Ratio |
|---|---|---|
| DSPC | 790.16 | 0.115 |
| DLin-MC3-DMA | 642.1 | 0.5 |
| DMG-PEG(2000) | 2509.2 | 0.0075 |
| Cholesterol* | 386.64 | 0.375 |
| DiO | 825.6 | 0.01 |

*Gal-Chol can be incorporated using the 25% and 50% LNP cholesterol mole fractions
** in some embodiments, Dio is omitted.

TABLE 5
LNP Formulation with SM-102

| SM-102 LNP | MW (mg/mL) | Molar Ratio |
|---|---|---|
| DSPC | 790.161 | 0.1 |
| SM-102 | 710.2 | 0.25 |
| DLin-MC3-DMA | 642.1 | 0.25 |
| DMG-PEG(2000) | 2509.2 | 0.015 |
| Cholesterol* | 386.64 | 0.375 |
| DiO** | 825.6 | 0.01 |

*Gal-Chol can be incorporated using the 25% and 50% LNP cholesterol mole fractions
**in some embodiments, Dio is omitted.

TABLE 6
LNP Formulation with ALC-0315

| SM-102 LNP | MW (mg/mL) | Molar Ratio |
|---|---|---|
| DSPC | 790.161 | 0.1 |
| ALC-0315 | 766.27 | 0.25 |
| DLin-MC3-DMA | 642.1 | 0.25 |
| DMG-PEG(2000) | 2509.2 | 0.015 |
| Cholesterol* | 386.64 | 0.375 |
| DiO** | 825.6 | 0.01 |

*Gal-Chol can be incorporated using the 25% and 50% LNP cholesterol mole fractions.
**in some embodiments, DiO is omitted.

TABLE 7
Summary of Oligonucleotides Encapsulated for Each Formulation

| Formulation | Oligonucleotides Encapsulated |
|---|---|
| LNP | pGFP, PCSK9, Cy3-GAPDH, AID, NRF2, |
| | HDAC2 |
| 25% Gal-Chol LNP | pGFP, PCSK9, Cy3-GAPDH, HDAC2 |
| 50% Gal-Chol LNP | pGFP, PCSK9, Cy3-GAPDH, HDAC2 |
| ½ PEG LNP | pGFP, PCSK9, Cy3-GAPDH, HDAC2 |
| (25% and 50% Gal-Chol) | |
| SM-102 LNP | pGFP, PCSK9, Cy3-GAPDH, HDAC2 |
| (25% and 50% Gal-Chol) | |
| ALC-0315 LNP | pGFP, PCSK9, Cy3-GAPDH, HDAC2 |
| (25% and 50% Gal-Chol) | |

TABLE 8
Summary of Encapsulated siRNA Constructs

| Target | Sequence | Notes |
|---|---|---|
| HDAC2 | Sense: 5′-GCCACUGCCGAAGAAAUGAtt-3′ (SEQ ID NO: 3) | siRNA |
| | Antisense 5′-UCAUUUCUUCGGCAGUGGCtt-3′ (SEQ ID NO: 4) | |
| GAPDH | Sense 5′-GCUCAUUUCCUGGUAUGACAACGAA-3′ | DsiRNA; |
| | (SEQ ID NO: 5) | Construct |
| | Antisense 5′-UUCGUUGUCAUACCAGGAAAUGAGCUU-3′ | contained 5′ |
| | (SEQ ID NO: 6) | Cyanine-3 tag |
| PCSK9 | Sense 5′-GGCAUUCAAUCCUCAGGUCtt-3′ (SEQ ID NO: 7) | siRNA |
| | Antisense 5′-GACCUGAGGAUUGAAUGCCtg-3′ (SEQ ID NO: 8) | |
| NRF2 | Sense 5′-UUUCUCCCAAUUCAGCCAGCCCAGC-3′ | DsiRNA |
| | (SEQ ID NO: 9) | |
| | Antisense 5′-GCUGGGCUGGCUGAAUUGGGAGAAAUU-3′ | |
| | (SEQ ID NO: 10) | |

TABLE 9
Effect of Lipid Ratio and % PEG in the Total Formulation and pKa of Lipid Combination over % EE and Transfection

| | | | | | | | | pKa of | | | Knock |
|---|---|---|---|---|---|---|---|---|---|---|---|
| | ionizable Lipid and Ratio | | | | Helper lipid ratio | | % PEG | lipid combo | DLS/ | [siRNA] | down |
| | Dlin:ALC:SM | Dlin | ALC-0315 | SM-102 | DSPC | DOPE | DMG-PEG | Ionizable | PDI | (ng/uL) | (%) |
| 1 | Dlin:ALC:SM | 4 | 0 | 0 | 10 | 0 | 1.5 | 6.44 | | | |
| 2 | Dlin:ALC:SM | 3 | 1 | 0 | 10 | 0 | 1.5 | 6.35 | | | |
| 3 | Dlin:ALC:SM | 2 | 2 | 0 | 10 | 0 | 1.5 | 6.27 | 200.3/0.01 | | |
| 4 | Dlin:ALC:SM | 1 | 3 | 0 | 10 | 0 | 1.5 | 6.18 | 215.4/0.117 | | |
| 5 | Dlin:ALC:SM | 0 | 4 | 0 | 10 | 0 | 1.5 | 6.09 | | | |
| 6 | Dlin:ALC:SM | 3 | 0 | 1 | 10 | 0 | 1.5 | 6.52 | | | |
| 7 | Dlin:ALC:SM | 2 | 0 | 2 | 10 | 0 | 1.5 | 6.60 | 241/0.049 | | |
| 8 | Dlin:ALC:SM | 1 | 0 | 3 | 10 | 0 | 1.5 | 6.67 | 178.9/0.089 | | |
| 9 | Dlin:ALC:SM | 0 | 0 | 4 | 10 | 0 | 1.5 | 6.75 | | | |
| 10 | Dlin:ALC:SM | 4 | 0 | 0 | 10 | 0 | 0.75 | 6.44 | | | |
| 11 | Dlin:ALC:SM | 0 | 4 | 0 | 8 | 2 | 1.5 | 6.09 | | | |

*all formulations shown can in some embodiments be made with 0, 25, and 50% galactosyl mole fractions according to the LNP formulations.

Different lipid combinations have been shown to have different transfection efficiencies relative to the cell type/line. Consequently, transfection efficiency can be optimized by modulating the lipid ratios, specifically the ionizable/cationic lipid ratios. Table 10 below presents exemplary LNP formulations that can be employed for encapsulating different nucleic acid types with optimized lipid combinations with respect to encapsulation and HepG2 transfection efficiency. DOPE can be included in the formulation((as shown) to aid in plasmid DNA encapsulation. In order to modulate the transfection efficiency for other cell lines the ratio of Dlin:ALC would be changed and screened against the specific cells/tissue.

TABLE 10
Exemplary LNP Formulations of the Presently Disclosed
Subject Matter for Different Nucleic Acids

| | Mol Fraction |
|---|---|
| Lipids for siRNA LNPs | |
| DSPC | 0.09 |
| Cholesterol | 0.28125 |
| Galactosyl-Cholesterol | 0.09375 |
| DMG-PEG (2000) | 0.025 |
| DLin-MC3-DMA | 0.167 |
| ALC-0315 | 0.333 |
| Lipids for pDNA LNPs | |
| DSPC | 0.09 |
| DOPE | 0.01 |
| Cholesterol | 0.28125 |
| Galactosyl-Cholesterol | 0.09375 |
| DMG-PEG (2000) | 0.025 |
| DLin-MC3-DMA | 0.167 |
| ALC-0315 | 0.333 |

### REFERENCES

All references listed in the instant disclosure, including but not limited to all patents, patent applications and publications thereof, scientific journal articles, and database entries (including but not limited to UniProt, EMBL, and GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.

Altschul et al. (1990a) Basic local alignment search tool. J Mol Biol 215:403-410.

Altschul et al. (1990b) Protein database searches for multiple alignments. Proc Natl Acad Sci USA 87:14:5509-13.

Altschul. et al. (1997) Gapped BLAST and PSI-BLAST: a new generation of protein database search programs. Nucleic Acids Res 25:3389-3402.

Arnold & Koenig (2022) PCSK9 Inhibitor Wars: How Does Inclisiran Fit in with Current Monoclonal Antibody Inhibitor Therapy? Considerations for Patient Selection. Curr Cardiol Rep 24(11):1657-1667.

Bird et al. (1988) Single-chain antigen-binding proteins. Science 242:423-426

Boyd et al. (2016) Increased Plasma PCSK9 Levels Are Associated with Reduced Endotoxin Clearance and the Development of Acute Organ Failures during Sepsis. Journal of Innate Immunity 8(2):211-220.

Devereux et al. (1984) A comprehensive set of sequence analysis programs for the VAX. Nucl Acids Res 12:387.

Gennaro (ed.) (1985) Remington's Pharmaceutical Sciences, Mack Publishing Co., Easton, Pennsylvania, United States of America.

Gennaro (ed.) (1990) Remington's Pharmaceutical Sciences, 18th ed., Mack Pub. Co., Easton, Pennsylvania, United States of America.

Gennaro (ed.) (2003) Remington: The Science and Practice of Pharmacy, 20th edition Lippincott, Williams & Wilkins, Philadelphia, Pennsylvania, United States of America.

Gross & Mienhofer (eds.) (1981) The Peptides, Vol. 3, Academic Press, New York, New York, United States of America. pages 3-88.

Gutschner & Diederichs (2012) The Hallmarks of Cancer. RNA Biol 9(6):703-719.

Harlow & Lane (1988) Antibodies, A Laboratory Manual, Cold Spring Harbor Publications, Cold Spring Harbor, New York, United States of America.

Flu et al. (2020) Therapeutic SiRNA: State of the Art, Signal Transduct Target Ther 5(1):1-25.

Huston et al. (1988) Protein engineering of antibody binding sites: recovery of specific activity in an anti-digoxin single-chain Fv analogue produced in Escherichia coli. Proc Natl Acad Sci USA 85:5879.

Jo et al. (2023) HDAC2 as a target for developing anti-cancer drugs. Computational and. Structural Biotechnology Journal 21:2048-2057.

Jones et al (1986) Replacing the complementarity-determining regions in a human antibody with those from a mouse. Nature 321:522.

Karlin & Altschul (1990) Methods for assessing the statistical significance of molecular sequence features by using general scoring schemes. Proc Natl Acad Sci USA 87(6):2264-2268.

Karlin & Altschul (1993) Applications and statistics for multiple high-scoring segments in molecular sequences. Proc Natl Acad Sci USA 90(12):5873-5877.

Lagace (2014) PCSK9 and LDLR Degradation: Regulatory Mechanisms in Circulation and in Cells. Curr Opin Lipidol 25(5):387-393.

Liu et al. (2021). Historic deacetylase-2: A potential regulator and therapeutic target in liver disease (Review). International Journal of Molecular Medicine 48(1):131.

Lodish et al. (2021) Molecular Cell Biology, Ninth Edition. Macmillan Learning, New York, New York, United States of America.

Mechtcheriakova et al. (2012) Activation-Induced Cytidine Deaminase (AID) Linking Immunity, Chronic Inflammation, and Cancer. Cancer Immunol Immunother 61(9):1591-1598.

Melesina et al. (2021). Strategies To Design Selective Histone Deacetylase Inhibitors. Chain Med. Chem 16(9):1336-1359.

Merćep et al. (2023) Present and Future of Dyslipidaemia Treatment-Review. J Clin Med 12(18):5839.

Mezu-Ndubuisi & Maheshwari (2021) The role of integrins in inflammation and angiogenesis. Pediatr Res 89:1619-1626.

Momtazi-Borojeni et al. (2019) PCSK9 and inflammation: a review of experimental and clinical evidence. Eur Heart J Cardiovasc Pharmacother 5(4):237-245.

Oikonomou & Antoniades (2019) The role of adipose tissue in cardiovascular health and disease. Nat Rev Cardiol 16:83-99.

Riechmann et al. (1988) Reshaping human antibodies for therapy. Nature 332(6162):323-327.

Roces et al. (2020) Manufacturing Considerations for the Development of Lipid Nanoparticles Using Microfluidics. Pharmaceutics 12(11):1095

Schüler et al. (2010). FIDAC2 attenuates TRAIL-induced apoptosis of pancreatic cancer cells. Molecular Cancer 9:80.

Sporn & Liby (2012) NRF2 and Cancer: The Good, the Bad and the Importance of Context. Nat Rev Cancer 12(8):564-571.

U.S. Patent Application Publication Nos. 2003/0017534, 2018/0298087, 2018/0312588, 2018/0346564, 2019/0151448.

U.S. Pat. Nos. 4,816,567; 5,482,856; 6,479,284; 6,677,436; 7,060,808; 7,906,625; 8.398.980; 8,436,150; 8,796,439; 10,253,111.

Walley et al. (2014) PCSK9 is a critical regulator of the innate immune response and septic shock outcome. Science Translational Medicine 6(258):258ra143.

Wang et al. (2023) Interactions between PCSK9 and NLRP3 inflammasome signaling in atherosclerosis. Front Immunol 14:1126823.

Wang et al. (2019) Adeno-Associated Virus Vector as a Platform for Gene Therapy Delivery. Nat Rev Drug Discov 18(5):358-378.

Winter & Milstein. (1991) Man-made antibodies. Nature 349(6307):293-299.

Zhang et al. (2015) Critical Protein GAPDH and Its Regulatory Mechanisms in Cancer Cells. Cancer Biol Med 12(1): 10-22.

Zhang et al. (2022) Modification of Lipid-Based Nanoparticles: An Efficient Delivery System for Nucleic Acid-Based Immunotherapy. Molecules 27(6):1943.

Zhu et al. (2.022) RNA-Based Therapeutics: An Overview and Prospectus. Cell Death Dis 13(7):1-15.

It will be understood that various details of the presently disclosed subject matter can be changed without departing from the scope of the presently disclosed subject matter, Furthermore, the foregoing description is for the purpose of illustration only, and not for the purpose of limitation.

1. A method for treating and/or preventing a disease, disorder, or condition associated with undesirable proprotein convertase subtilisin/kexin type 9 (PCSK9) gene expression, the method comprising administering to a subject in need thereof an effective amount of a comprising a sugar-conjugated lipid nanoparticle (LNP) and an active agent encapsulated therein, optionally wherein the sugar is a hexose monosaccharide, wherein the active agent modulates the PCSK9 gene expression to thereby treat and/or prevent the disease, disorder, or condition.

2. The method of claim 1, wherein the sugar is selected from the group consisting of mono-, di-, and triannary galactosyl, N-acetyl galactosamine (GalNac), glucose, N-acetyl glucosamine (GluNac), mannose, trehalose, fucoidan, pyranose, and furanose.

3. The method of claim 1, wherein the LNP is a galactosyl-conjugated LNP, and the galactosyl-conjugated LNP further comprises a lipid component comprising about 5-10% cholesterol.

4. The method of claim 1, wherein the LNP, optionally the galactosyl-conjugated LNP, comprises a lipid component comprising one or more of hydrolyzable ionizable lipids, optionally selected from the group consisting of D-Lin-MC3-DMA, ALC-0315, and SM-102; cholesterol and/or an analog thereof, optionally β-sitosterol; and optionally one or more helper lipids, optionally selected from the group consisting of DSPC, DOPE, and DMG-2000-PEG, further optionally two, three, or all four of D-Lin-MC3-DMA, cholesterol, DSPC, and DMG-2000-PEG.

5. The method of claim 1, wherein the LNP is a galactosyl-conjugated LNP that comprises one or more galactosyl moieties bioconjugated to cholesterol present with a lipid component of the galactosyl-conjugated LNP.

6. The method of claim 1, wherein the active agent is a nucleic acid, optionally an inhibitory nucleic acid, further optionally an siRNA.

7. The method of claim 6, wherein the inhibitory nucleic acid inhibits a biological activity of a PCSK9 gene product, optionally a human PCSK9 gene product, and further optionally wherein the inhibitory nucleic acid is an inclisiran derivative lacking a GalNAc moiety.

8. The method of claim 1, wherein the LNP, optionally the galactosyl-conjugated LNP, targets the active agent to a cell, tissue, or organ of interest, optionally wherein the cell tissue, or organ of interest is selected from the group consisting of vasculature or a vasculature cell, optionally a vascular endothelial cell, a muscle cell, a fibroblast, and/or a non-vascular endothelial cell.

9. The method of any one of claims 1-8, wherein the LNP comprises a ratiometric combination of ionizable lipids.

10. The method of claim 9, wherein the LNP comprises a ratiometric combination of ionizable lipids with a specific dissociation constant value of between about 6.05 and about 6.44.

11. The method of claim 1, wherein the LNP comprises one or more mono-, di-, and/or ternary hydrophobic fatty acids and/or one or more hydrophilic polyethylene glycols conjugated to the sugar moiety onto cholesterol at its head or tail and/or onto a polyethylene glycol (PEG) moiety.

12. The method of claim 11, wherein at least one of the one or more fatty acids and/or the PEG moiety comprises a chain length of 1 to 15 carbons.

13. The method of claim 1, wherein the disease, disorder, or condition is selected from the group consisting of an inflammatory disease, disorder, or condition; a cardiovascular disease, disorder, or condition, optionally atherosclerosis and/or thrombosis; and sepsis, septic shock, cytokine storm, or sequelae thereof.

14. The method of claim 13, further comprising administering to the subject in need thereof an effective amount of a second active agent that is appropriate for treating the disease, disorder, or condition.

15. The method of claim 14, wherein the disease, disorder, or condition is:
(i) an inflammatory disease, disorder, or condition, and the second active agent is an anti-inflammatory agent, optionally a nonsteroidal anti-inflammatory drug (NSAID), a steroid, acetaminophen or other COX-3 inhibitor, a 5-lipoxygenase inhibitor, a leukotriene receptor antagonist, a leukotriene A4 hydrolase inhibitor, an antihistaminic, a histamine 2 receptor antagonist, a phosphodiesterase-4 antagonist, a cytokine antagonist, a CD44 antagonist, an antineoplastic agent, a 3-hydroxy-3-methylglutaryl coenzyme A inhibitor (e.g., a statin), an estrogen, an androgen, an antiplatelet agent, an antidepressant, a Helicobacter pylori inhibitor, a proton pump inhibitor, a thiazolidinedione, or a dual-action compound;
(ii) a cardiovascular disease, disorder, or condition, optionally atherosclerosis, and the second active agent is an acyl-coenzyme A: cholesterol acyltransferase (ACAT) inhibitor, including but not limited to a selective inhibitor of ACAT-1, ACAT-2 as well as dual inhibitors of ACAT-1 and ACAT-2; an alpha-adrenergic blocking drug (alpha-blocker), an alpha/beta blocker, an angiotensin-converting enzyme (ACE) inhibitor, an aldosterone antagonist, an angiotensin II receptor antagonist, an anti-arrhythmic, an anticoagulant, an antiplatelet agent, an apolipoprotein A-1 (apoA-1) mimetic, a beta-blocker, a bile acid sequestrant, a calcium-channel blocker, an ApoB cholesteryl ester transfer protein (CETP) inhibitor, a cholesterol absorption inhibitor, a diuretic, a dyslipidemia agent, an endothelin receptor antagonist, a fibrate, a 3-hydroxy-3-methyl-glutaryl-coenzyme A (HMG-CoA) reductase inhibitor, an LCAT activator, an LDL receptor inducer, a lipase inhibitor, a lipoprotein-associated phospholipase A2 (Lp-PLA2) inhibitor, a microsomal triglyceride transfer protein (MTP) inhibitor, a platelet aggregation inhibitor, a PPAR agonist and/or activator, optionally a PPARγ agonist, PPARα agonist, and/or a PPAR dual α/γ agonists, a second PCSK9 antisense nucleic acid or RNAi, a squalene epoxidase inhibitor, a squalene synthetase inhibitor, a thrombolytic, a thyroid receptor beta activator, or any combination thereof;
(iii) a cardiovascular disease, disorder, or condition, and the second active agent targets a vasculature cell, a muscle cell, a fibroblast, and/or an endothelial cell, optionally a vascular endothelial cell, wherein the second active agent binds to a target selected from the group consisting of LOX-1, a selectin, optionally a P-selectin or an E-selectin, an integrins, and a neuropilin; and/or
(iv) sepsis, septic shock, or cytokine storm and the second active agent is an antibiotic, a complement inhibitor, optionally a complement inhibitor selected from the group consisting of a C5a inhibitor, a C5aR inhibitor, a C3 inhibitor, optionally wherein the C3 inhibitor is Compstatin, a Compstatin analog, a Compstatin peptidomimetic, or a Compstatin derivative, a factor D inhibitor, a factor B inhibitor, a C4 inhibitor, a C1q inhibitor, or any combination thereof.

16. A method for targeting an active agent to a hepatocyte, the method comprising contacting the hepatocyte with a composition comprising an active agent encapsulated by a sugar-conjugated lipid nanoparticle (LNP), optionally a hexose monosaccharide-conjugated LNP, further optionally an N-acetylgalactosamine (GalNAc) conjugated lipid nanoparticle (LNP), wherein the sugar-conjugated LNP comprises a lipid component comprising ionizable aminolipid ALC-0315 (6-((2-hexyldecanoyl)oxy)-N-(6-((2-hexyldecanoyl)oxy)hexyl)-N-(4-hydroxybutyl)hexan-1-aminium) and one or more of D-Lin-MC3-DMA, cholesterol, DSPC, DOPE, and DMG-2000-PEG, optionally two, three, four, or all five of D-Lin-MC3-DMA, cholesterol, DSPC, and DMG-2000-PEG, further optionally wherein the lipid component comprises cholesterol with one or more GalNAc moieties conjugated thereto, optionally with 25% of the cholesterol being galactosyl-cholesterol, whereby the active agent is targeted to the hepatocyte.

17-22. (canceled)


SEQUENCE LISTING (10 sequences)

SEQ ID NO: 1 - RNA, 21 nt, synthetic construct
  source 1..21: mol_type = other RNA
ctagacctgttttgcttttgt

SEQ ID NO: 2 - RNA, 23 nt, synthetic construct
  source 1..23: mol_type = other RNA
acaaaagcaaaacaggtctagaa

SEQ ID NO: 3 - RNA, 21 nt, synthetic construct
  source 1..21: mol_type = other RNA
gccactgccgaagaaatgatt

SEQ ID NO: 4 - RNA, 21 nt, synthetic construct
  source 1..21: mol_type = other RNA
tcatttcttcggcagtggctt

SEQ ID NO: 5 - RNA, 25 nt, synthetic construct
  source 1..25: mol_type = other RNA
gctcatttcctggtatgacaacgaa

SEQ ID NO: 6 - RNA, 27 nt, synthetic construct
  source 1..27: mol_type = other RNA
ttcgttgtcataccaggaaatgagctt

SEQ ID NO: 7 - RNA, 21 nt, synthetic construct
  source 1..21: mol_type = other RNA
ggcattcaatcctcaggtctt

SEQ ID NO: 8 - RNA, 21 nt, synthetic construct
  source 1..21: mol_type = other RNA
gacctgaggattgaatgcctg

SEQ ID NO: 9 - RNA, 25 nt, synthetic construct
  source 1..25: mol_type = other RNA
tttctcccaattcagccagcccagc

SEQ ID NO: 10 - RNA, 27 nt, synthetic construct
  source 1..27: mol_type = other RNA
gctgggctggctgaattgggagaaatt